(19) United States
(12) Patent Application Publication
(10) Pub. No.: US 2008/0287529 A1
(43) Pub. Date: Nov. 20, 2008

(54) CRYSTAL STRUCTURES OF SGLT2 INHIBITORS AND PROCESSES FOR PREPARING SAME

(75) Inventors: Prashant P. Deshpande, Princeton, NJ (US); Lifen Lori Shen, Wayne, PA (US); Jack Z. Gougoutas, Princeton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/120,722

(22) Filed: May 15, 2008

Related U.S. Application Data
(60) Provisional application No. 60/938,731, filed on May 18, 2007.

(51) Int. Cl. A61K 31/351; C07D 315/00; A61P 3/00

(52) U.S. Cl. 514/460; 549/417

(57) ABSTRACT

The present invention relates to physical crystal structures of compound of the formula (I):

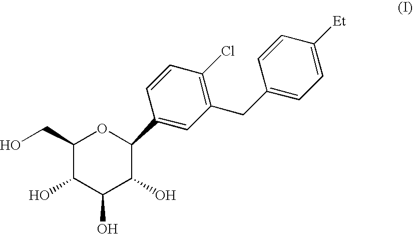

which is an H-1 form, H-2 form or S-PG form, pharmaceutical compositions containing structures of compound I and methods of treating diseases using compound I.

### FIELD OF THE INVENTION

The present invention relates to crystal structures of SGLT2 Inhibitors, pharmaceutical compositions thereof, processes for preparing such crystal structures, and methods of treating disorders therewith.

### BACKGROUND OF THE INVENTION

Approximately 100 million people worldwide suffer from type II diabetes (NIDDM), which is characterized by hyperglycemia due to excessive hepatic glucose production and peripheral insulin resistance, the root causes for which are as yet unknown. Consistent control of plasma glucose levels in diabetes patients may offset the development of diabetic complications and beta cell failure seen in advanced disease.

Plasma glucose is normally filtered in the kidney in the glomerulus and actively reabsorbed in the proximal tubule. Ninety percent of glucose reuptake in the kidney occurs in the epithelial cells of the early S1 segment of the renal cortical proximal tubule. SGLT2, a 672 amino acid protein containing 14 membrane-spanning segments that is predominantly expressed in the early S1 segment of the renal proximal tubules, is likely to be the major transporter responsible for this reuptake. The substrate specificity, sodium dependence, and localization of SGLT2 are consistent with the properties of the high capacity, low affinity, sodium-dependent glucose transporter previously characterized in human cortical kidney proximal tubules. In addition, hybrid depletion studies implicate SGLT2 as the predominant Na⁺/glucose cotransporter in the S1 segment of the proximal tubule, since virtually all Na-dependent glucose transport activity encoded in mRNA from rat kidney cortex is inhibited by an antisense oligonucleotide specific to rat SGLT2. In humans, mutations in SGLT2 have been associated with familial structures of renal glucosuria, providing further evidence of the primary role of SGLT2 in renal glucose reabsorption. In such patients, renal morphology and renal function is otherwise normal. Inhibition of SGLT2 would be predicted to reduce plasma glucose levels via enhanced glucose excretion in diabetic patients.

Selective inhibition of SGLT2 in diabetic patients could normalize plasma glucose by enhancing the excretion of glucose in the urine, thereby improving insulin sensitivity, and delaying the development of diabetic complications, in the absence of significant gastrointestinal side effects.

### SUMMARY OF THE INVENTION

The present invention relates to crystal structures of a compound of the formula I

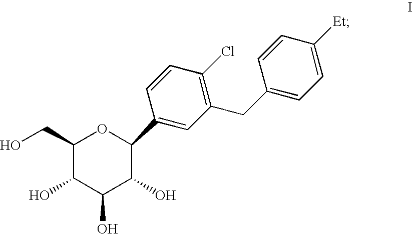

pharmaceutical compositions containing crystal structures of compound I, including the (S)-propylene glycol ((S)—PG) structure Ia

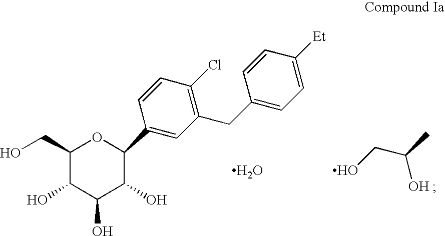

the L-phenylalanine (L-Phe) structure (form H-1)

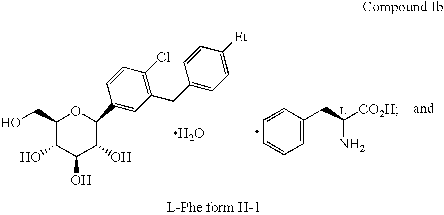

the L-Phe structure (form H-2)

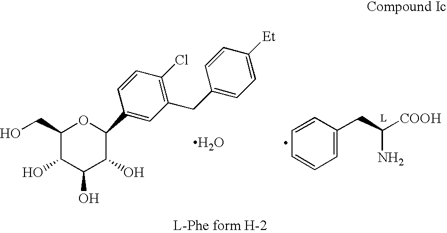

processes for preparing such crystal structures; and methods of treating diabetes and related diseases using the crystal structures of the compound I.

The compound of formula I in the form of a non-crystalline solid is disclosed in U.S. application Ser. No. 11/233,617 (Washburn et al.) filed Sep. 23, 2005, the disclosure of which in its entirety is incorporated herein by reference. A genus of compounds which encompass the compound of formula I is disclosed in U.S. Pat. No. 6,774,112 to Gougoutas, the disclosure of which in its entirety is incorporated herein by reference.

In addition, in accordance with the present invention, a process for the preparation of the crystalline compound (S)—PG of the structure Ia is provided

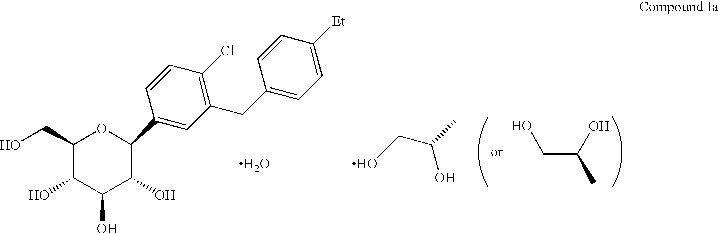

which includes the steps of dissolving a compound I (prepared as described in U.S. application Ser. No. 11/233,617 filed Sep. 23, 2005, Example 1), of the structure

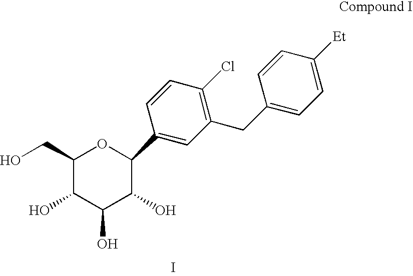

in a water-miscible organic solvent such as methyl t-butyl ether, treating the resulting solution with (S)-propylene glycol in an organic solvent such as methyl t-butyl ether, optionally adding seeds of compound Ia ((S)-PG) to the reaction mixture, and forming crystals of compound Ia ((S)—PG).

In still another aspect of the present invention, a novel process is provided for preparing compound Ia

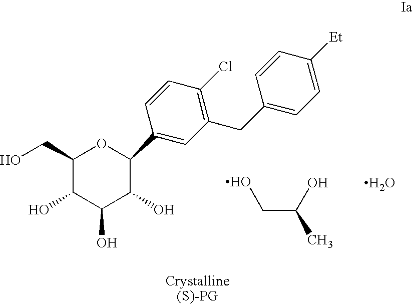

in a telescoped reaction, which includes the steps of reducing a compound B of the structure

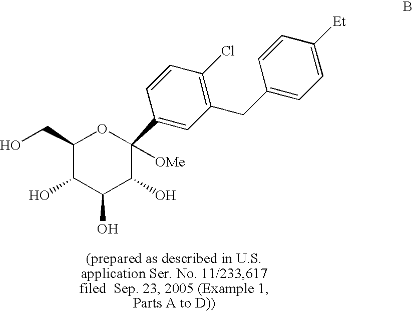

to remove the methoxy group by treating compound B with a reducing agent, such as triethylsilyl hydride and an activating group such as BF₃.Et₂O, and an organic solvent such as CH₃CN, and water, separating out the compound of the structure I

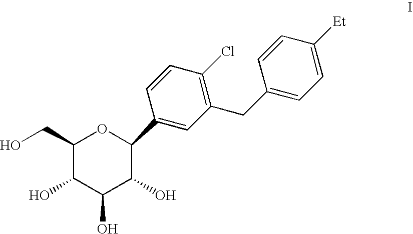

and treating compound I with (S)-propylene glycol in the presence of a solvent such as methyl t-butyl ether, optionally with seeds of compound Ia ((S)—PG), to form a crystal slurry of compound Ia ((S)—PG), and separating out compound Ia ((S)—PG).

The above process of the invention is a telescoped or one-pot operation which minimizes the production of intermediates, resulting in improved yield and priority of the final crystalline compound Ia.

The crystalline compound Ia which is also referred to as the (S)-propylene glycol solvate of compound I is a novel crystalline structure and is part of the present invention.

In yet another embodiment of the invention, a process is provided for forming the L-phenylalanine of the structure of formula Ic form H-2

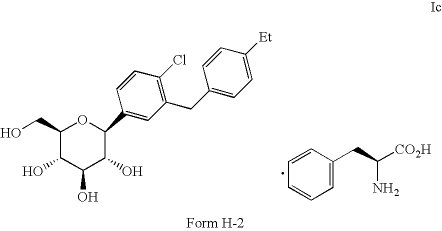

which includes the steps of dissolving compound A

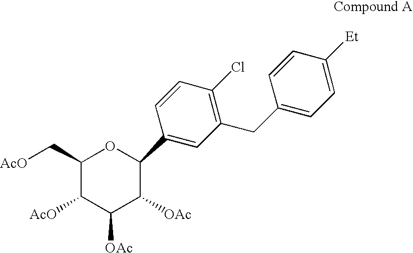

in a water-miscible organic solvent such as isopropyl alcohol, cooling the resulting solution, adding water to the solution, adding an acid to the solution to neutralize the solution, adding L-phenylalanine, isopropyl alcohol and water to the solution, optionally adding seeds of structure Ic form H-2 in a slurry with solvent such as isopropyl alcohol and water, and cooling the slurry to form crystals of structure Ic form H-2.

In the process for preparing structure Ic form H-2 described above, crystals of structure Ib form H-1 may be formed as well.

The preparation of compounds of formula I is generally described in U.S. Pat. No. 6,414,126, and specifically described in U.S. application Ser. No. 11/233,617, filed Sep. 23, 2005.

### BRIEF DESCRIPTION OF THE FIGURES

The invention is illustrated by reference to the accompanying drawings described below.

FIG. 1 shows calculated (simulated at 25° C.) and observed (experimental at room temperature) powder X-ray diffraction patterns of the H-2 crystal structure Ic.

FIG. 2 shows hybrid (room temperature) and observed (experimental at room temperature) powder X-ray diffraction patterns of the (S)—PG crystal structure Ia.

FIG. 3 shows a differential scanning calorimetry thermogram of the H-2 crystal structure Ic.

FIG. 4 shows a differential scanning calorimetry thermogram of the (S)—PG crystal structure Ia.

FIG. 5 shows a thermogravimetric analysis curve of the H-2 crystal structure Ic.

FIG. 6 shows a thermogravimetric analysis curve of the (S)—PG crystal structure Ia.

FIG. 7 shows a moisture-sorption isotherm analysis of the H-2 crystal structure Ic.

FIG. 8 shows ¹³C NMR CPMAS spectrum for the (S)—PG crystal structure Ia.

### DETAILED DESCRIPTION OF THE INVENTION

The present invention provides, at least in part, crystal structures of compound I as a novel material, in particular in pharmaceutically acceptable form. Three crystal structures, H-1 (Ib), H-2 (Ic) and (S)—PG (Ia) of compound I have been isolated and/or identified.

The term “pharmaceutically acceptable”, as used herein, refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. In certain preferred embodiments, Compounds I, Ia, Ib and/or Ic may be in substantially pure form. The term “substantially pure”, as used herein, means a compound having a purity greater than about 90% including, for example, about 91%, about 92%, about 93%, about 94%, about 95%, about 96%, about 97%, about 98%, about 99%, and about 100%.

The ability of a compound to exist in different crystal structures is known as polymorphism. As used herein “polymorph” refers to crystal structures having the same chemical composition but different spatial arrangements of the molecules, atoms, and/or ions forming the crystal. While polymorphs have the same chemical composition, they differ in packing and geometrical arrangement, and may exhibit different physical properties such as melting point, shape, color, density, hardness, deformability, stability, dissolution, and the like. Depending on their temperature-stability relationship, two polymorphs may be either monotropic or enantiotropic. For a monotropic system, the relative stability between the two solid phases remains unchanged as the temperature is changed. In contrast, in an enantiotropic system there exists a transition temperature at which the stability of the two phases reverse. (Theory and Origin of Polymorphism in Polymorphism in Pharmaceutical Solids (1999) ISBN:)-8247-0237).

Samples of the crystal structures may be provided with substantially pure phase homogeneity, indicating the presence of a dominant amount of a single crystal structure and optionally minor amounts of one or more other crystal structures. The presence of more than one crystal structure in a sample may be determined by techniques such as powder X-ray diffraction (PXRD) or solid state nuclear magnetic resonance spectroscopy (SSNMR). For example, the presence of extra peaks in the comparison of an experimentally measured PXRD pattern (observed) with a simulated PXRD pattern (calculated) may indicate more than one crystal structure in the sample. The simulated PXRD may be calculated from single crystal X-ray data. (see Smith, D. K., “A FORTRAN Program for Calculating X-Ray Powder Diffraction Patterns,” Lawrence Radiation Laboratory, Livermore, Calif., UCRL-7196, April 1963; see also Yin, S. et al., American Pharmaceutical Review, 6(2):80 (2003)). Preferably, the crystal structure has substantially pure phase homogeneity as indicated by less than 10%, preferably less than 5%, and more preferably less than 2% of the total peak area in the experimentally measured PXRD pattern arising from the extra peaks that are absent from the simulated PXRD pattern. Most preferred is a crystal structure having substantially pure phase homogeneity with less than 1% of the total peak area in the experimentally measured PXRD pattern arising from the extra peaks that are absent from the simulated PXRD pattern.

The various structures described herein may be distinguishable from one another through the use of various analytical techniques known to one of ordinary skill in the art. Such techniques include, but are not limited to, solid state nuclear magnetic resonance (SSNMR) spectroscopy, X-ray powder diffraction (PXRD), differential scanning calorimetry (DSC), and/or thermogravimetric analysis (TGA).

### Preparation of Crystal Structures

Procedures for the preparation of crystal structures are known in the art. The crystal structures may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystal structures from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (counter solvents) to the solvent mixture. High throughput crystallization techniques may be employed to prepare crystal structures including polymorphs.

Crystals of drugs, including polymorphs, methods of preparation, and characterization of drug crystals are discussed in Byrn, S. R. et al., Solid-State Chemistry of Drugs, 2^{nd} Edition, SSCI, West Lafayette, Ind. (1999).

Seed crystals may be added to any crystallization mixture to promote crystallization. As will be clear to the skilled artisan, seeding is used as a means of controlling growth of a particular crystal structure or as a means of controlling the particle size distribution of the crystalline product. Accordingly, calculation of the amount of seeds needed depends on the size of the seed available and the desired size of an average product particle as described, for example, in Mullin, J. W. et al., “Programmed cooling of batch crystallizers,” Chemical Engineering Science, 26:369-377 (1971). In general, seeds of small size are needed to effectively control the growth of crystals in the batch. Seeds of small size may be generated by sieving, milling, or micronizing of larger crystals, or by micro-crystallization of solutions. Care should be taken that milling or micronizing of crystals does not result in any change in crystallinity from the desired crystal structure (i.e., change to amorphous or to another polymorph).

As used herein, the term “room temperature” or “RT” denotes an ambient temperature from 20 to 25° C. (68-77° F.).

The crystalline compound of the structure Ia ((S)—PG) SC-3 of the invention prepared according to the following telescoped reaction as shown in Scheme I:

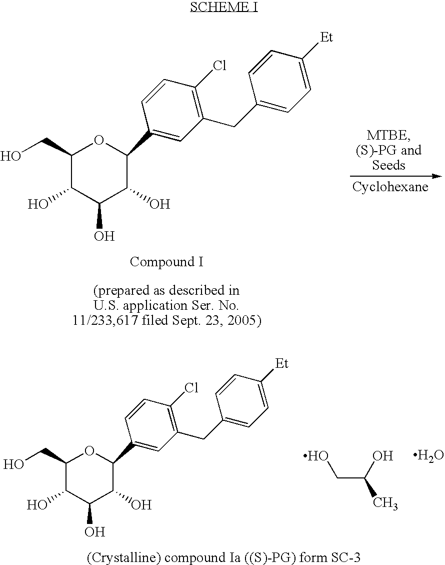

Referring to Scheme I, compound I in the form of an amorphous solid or crystalline solid is dissolved in a water-miscible organic solvent such as methyl t-butyl ether (MTBE) to form a solution and the resulting solution is treated with (S)-propylene glycol ((S)—PG) in an organic polar solvent such as an alkyl acetate, for example ethyl acetate, methyl acetate and isopropyl acetate, and methyl t-butyl ether, preferably, methyl t-butyl ether (MTBE). Optionally seeds of compound ((S)—PG) Ia are added to the reaction mixture. A crystal slurry of compound ((S)—PG) Ia forms which is separated from the crystal slurry. The crystalline compound Ia may be separated from the slurry employing conventional procedures, for example, the slurry of compound Ia is treated with an organic solvent such as cyclohexane, and crystalline compound Ia is recovered.

The seeds of crystalline compound Ia may be prepared as described with respect to Scheme I, with or without the seeding step.

In carrying out the above telescoped reaction of Scheme I, (S)-propylene glycol ((S)—PG) will be employed in a molar ratio to compound I within the range from about 0.8:1 to about 1.2:1, preferably from about 0.9:1 to about 1:1.

In another aspect of the invention, as described hereinbefore, compound I may be prepared in a telescoped reaction wherein compound B is reacted with a reducing agent such as a silyl hydride including alkylsilyl hydrides, preferably a trialkylsilyl hydride such as triethylsilyl hydride, preferably in the presence of an activating group including a Lewis acid such as BF₃.Et₂O or BF₃.2CH₃COOH, for example, and a reaction solvent such as CH₃CN mixtures of CH₃CN/toluene, or mixtures of CH₃CN/dichloromethane, at ambient temperatures (e.g., 15° C.).

The crystalline compound of the structure Ia ((S)—PG) of the invention may also be prepared according to the reaction Scheme II set out below:

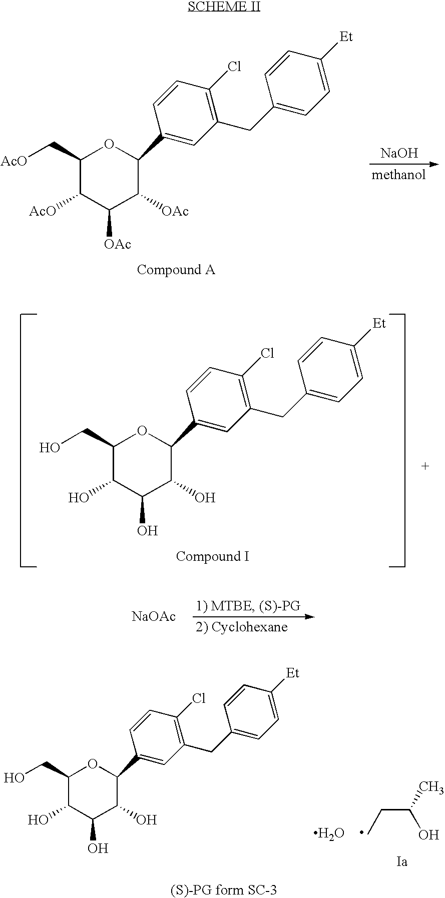

wherein compound A is treated with an alcohol solvent such as methanol or ethanol, preferably methanol, water and aqueous base such as an alkali metal hydroxide such as NaOH, KOH or LiOH, preferably NaOH, preferably under an inert atmosphere such as nitrogen, at an elevated temperature within the range from about 50 to about 85° C., preferably from about 60 to about 80° C., to form compound I.

The aqueous base will be employed in a molar ratio of compound A within the range from about 0.25:1 to about 1:1, preferably from about 3:1 to about 5:1.

To form compound Ia, as shown in Scheme I, the reaction mixture containing compound I is treated with an organic polar solvent such as methyl t-butyl ether (MTBE), or an alkyl acetate as set out above with respect to Scheme I, preferably MTBE, to separate out compound I which is treated with (S)-propylene glycol to form a thick slurry containing crystalline product Ia (S)—PG. The crystalline compound Ia is separated from the slurry employing conventional procedures, for example, the slurry of compound Ia is treated with an organic solvent such as cyclohexane:MTBE, isooctane:MTBE, heptane:MTBE, cyclohexane, an alkyl acetate as set out with respect to Scheme I, preferably cyclohexane:MTBE, and crystalline compound Ia is recovered.

Referring to Scheme III, the L-phenylalanine structure H-2 is prepared as follows:

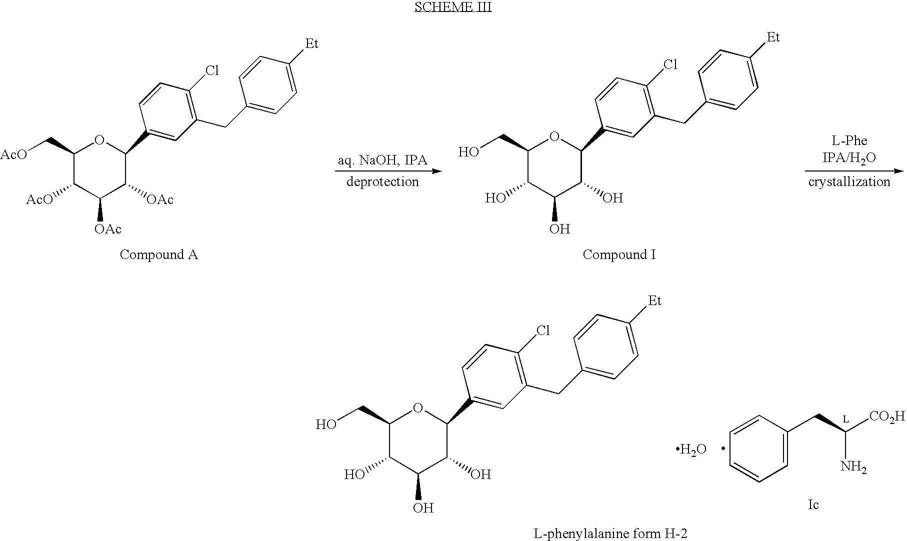

Compound A is dissolved in an alcohol solvent such as aqueous isopropyl alcohol (IPA), ethanol or methanol, preferably aqueous isopropyl alcohol, treated with strong base such as NaOH or KOH, and heated at a temperature within the range from about 40 to about 60° C., preferably from about 45 to about 55° C., for a period from about 10 to about 60 minutes, preferably from about 25 to about 30 minutes. The mixture is cooled to a temperature with the range from about 15° C. to about 30° C., preferably from about 20° C. to about 25° C. The cooled mixture is neutralized with strong mineral acid such as HCl to a pH within the range from about 5.5 to about 7.5, preferably from about 6 to about 6.5. L-Phenylalanine (L-Phe) is added to the neutralized mixture and additional water and IPA are added, if necessary, to adjust solvent composition to within the range from about 14 to about 24 vol % IPA. The resulting mixture is heated at a temperature within the range from about RT to about 80° C., preferably from about 60 to about 75° C., to obtain a clear solution which is cooled to a temperature within the range from about 40 to about 55° C., preferably from about 47 to about 52° C. In a preferred embodiment, a slurry of seeds of compound Ic H-2 (prepared as described herein in Scheme II with or without the seeding step) in aqueous alcohol solvent such as IPA/H₂O is added. The resulting slurry is cooled to a temperature within the range from about 30 to about 45° C., preferably from about 35 to about 45° C., from about 1 to about 4 hours, preferably from about 2 to about 3 hours, to form crystalline compound Ia H-2. Seeds of the L-phenylalanine crystal form Ic H-2 in aqueous alcohol solvent (containing from about 0.3% to about 1%, preferably from about 0.5% seeds) are added to the above slurry. The slurry is cooled and L-phenylalanine crystals Ic form H-2 are covered.

### EXAMPLES

The following examples are provided to describe the invention in further detail. These examples, which set forth the best mode presently contemplated for carrying out the invention, are intended to illustrate and not to limit the invention.

### Example 1

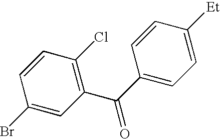

### A. 5-Bromo-2-chloro-4′-ethylbenzophenone

To a 2L round bottom flask containing a magnetic stirred suspension of commercial 5-bromo-2-chlorobenzoic acid (410 g, 1.74 mol) in 700 mL of CH₂Cl₂ was added oxalyl chloride (235 g, 1.85 mol) followed by 1.5 mL of DMF. To trap the resultant HCl, the flask was fitted with tubing so that the gas was discharged above the surface of a stirred aq KOH solution. When the vigorous evolution of gas ceased after two hours, the homogeneous reaction was stirred overnight prior to removal of the volatiles under vacuum using a rotary evaporator. The resultant oil solidified during subsequent evacuation.

After dissolving the crude 5-bromo-2-chlorobenzoyl chloride in 530 ml of ethylbenzene, the yellow solution was cooled to −3° C. prior to adding AlCl₃ (257 g, 1.93 mol) in ˜30 g portions over 60 min to insure that the temperature did not exceed 10° C. The copious amounts of HCl gas which began to evolve after 60% of the AlCl₃ had been added were trapped by passing the gas over a stirred conc. NaOH solution. If the reaction were more concentrated, a magnetic stirrer could not have maintained stirring upon completion of the addition of AlCl₃. After stirring for 1 hr as the bath warmed to ˜15° C., the bath was removed. After 4 hr at 20° C., the thick syrup was poured over ice (1.5 kg). Subsequently, once the stirred suspension had cooled, H₂O (1 L) was added prior to being extracted 4× with EtOAc. The combined organic extracts were washed 2× with 1N HCl, 3× with 1M KOH, and 2× with brine prior to drying over Na₂SO₄. The volatiles were removed using first a rotary evaporator and then by heating at ˜60° C. at 1 Torr. ¹H NMR analysis of the resultant dark oil revealed the residue to be a 1:14 mixture of ortho/para isomers. Dissolution in hexane and followed by filtration through a silica gel pad removed most of the color. Concentration of the eluent yielded 560 g (99%) of a 14:1 mixture of 5-bromo-2-chloro-4′-ethylbenzophenone/5-bromo-2-chloro-2′-ethylbenzophenone.

HPLC retention time: 4.7 min, YMC S5 C-18 4.6×50 mm column, 2.5 mL/min, detection at 220 nM; 4 min gradient 0-100% B hold 2 min at 100% B. Solvent A: 10% MeOH/H₂O+0.2% H₃PO₄. Solvent B: 90% MeOH/H₂O+0.2% H₃PO₄.

### 5-Bromo-2-chloro-4′-ethylbenzophenone

¹H NMR (400 MHz, CDCl₃) δ 7.73 (d, 2H, J_{AB}=8.2 Hz), 7.54 (dd, 1H, J=2.2 Hz, J=8.8 Hz), 7.32 (d, 1H, J=8.8 Hz), 7.295 (d, 2H, J_{AB}=8.2 Hz), 2.72 (q, 2H, J=7.7 Hz), 1.27 (t, 3H, J=7.7 Hz).

¹³C NMR (100 MHz, CDCl₃) δ 193.13. 151.33, 140.49, 133.8, 133.52, 131.6, 131.44, 130.34, 130.16, 128.28, 120.44, 29.04, 15.02.

### 5-Bromo-2-chloro-2′-ethylbenzophenone (Distinctive Signals)

¹H NMR (400 MHz, CDCl₃) δ 2.64 (q, 2H, J=7.7 Hz), 1.23 (t, 3H, J=7.7 Hz).

¹³C NMR (100 MHz, CDCl₃) δ 28.9, 15.5.

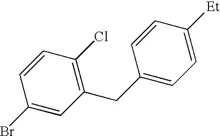

### B. 5-Bromo-2-chloro-4′-ethyldiphenylmethane

To a stirred solution of Et₃SiH (400 g, 3.45 mol and 5-bromo-2-chloro-4′-ethylbenzophenone (534 g, 1.65 mol) containing ˜7% of the isomeric ketone in 300 mL of TFA at 30° C. was added CF₃SO₃H (1.5 g, 0.01 mol). Within minutes the temperature increased causing the solution to reflux violently. Caution: this moderate exotherm requires cooling with an external ice bath. After 1 hr, HPLC revealed the reaction to be 90% complete. After addition of an additional Et₃SiH (20 g ) and heating overnight at 70° C., the reaction was >95% complete by HPLC analysis. Upon cooling, the volatiles were removed by bulb to bulb distillation at reduced pressure. The resultant ˜1 L of light gray oil was poured into 1 L of H₂O. The mixture was extracted 3× with hexane; the combined organic layers were washed 3× with H₂O, 2× with aq Na₂CO₃ and 2× with brine before drying over Na₂SO₄. After concentration using a rotary evaporator, ˜1 L of clear light amber oil remained. This material was further concentrated; the (Et₃Si)₂O (450 mL) was removed by distillation at 0.6 Torr. Once the temperature at the distillation head reached 75° C., the pot was allowed to cool. ¹H NMR analysis of the pot revealed it to contain an ˜8:1 mixture of diarylmethane to (Et₃Si)₂O. Crystallization of this mixture was achieved by pouring the product into vigorously stirred 10° C. 85% EtOH/H₂O (1.2 L), After stirring for several hours, the crystals were collected by filtration, washed with cold 1:1 EtOH/H₂O and dried under vacuum. The 5-bromo-2-chloro-4′-ethyldiphenylmethane (500 g), obtained as a low melting solid containing ˜1% (Et₃Si)₂O, was used without further purification.

HPLC retention time: 5.3 min, YMC S5 C-18 4.6×50 mm column, 2.5 mL/min, detection at 220 nM; 4 min gradient 0-100% B hold 2 min at 100% B. Solvent A: 10% MeOH/H₂O+0.2% H₃PO₄. Solvent B: 90% MeOH/H₂O+0.2% H₃PO₄.

¹H NMR (125 MHz, CDCl₃) δ 7.27-7.23 (m, 3H), 7.14 (d, 2H, J_{AB}=7.7 Hz), 7.09 (d, 2H, J_{AB}=7.7 Hz), 2.63 (q, 2H, J=7.7 Hz), 1.23 (t, 3H, J=7.7 Hz).

¹³C NMR (100 MHz, CDCl₃) δ 142.46. 141.08, 135.68, 133.64, 133.13, 130.85, 130.55, 128.83, 128.1, 120.0, 38.62, 28.43, 15.51.

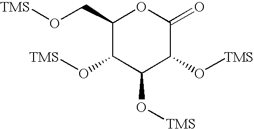

### C. 2,3,4,6-Tetra-O-trimethylsilyl-D-glucolactone

To a stirred −5° C. solution of gluconolactone (239 g, 1.34 mol) and N-methylmorpholine (1180 mL, 10.73 mol) in 2.4 L of THF under Ar was added trimethylsilyl chloride (1022 mL, 8.05 mol) via dropping funnel at a rate such that the temperature did not exceed 5° C. After 1 hr the stirred reaction was heated to 35° C. for 5 hr whereupon it was allowed to cool to 20° C. as the reaction stirred overnight. After dilution with 3.6 L of toluene, the mixture was cooled to 0-5° C. prior to cautiously adding 7 L of H₂O at a rate such that the temperature did not exceed 10° C. Note a severe exotherm results upon addition of the first portion of H₂O. After mixing, the phases were allowed to separate and then split. The organic phase was washed with aq. NaH₂PO₄ (2 L), H₂O (1 L), and brine (1 L). The organic layer was then concentrated under vacuum using a rotary evaporator; the resultant light yellow oil was twice taken up 250 mL of toluene and reconcentrated to yield 616 g.

### D.

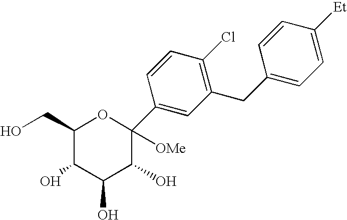

To a stirred −78° solution of Part B 5-bromo-2-chloro-4′-ethyldiphenylmethane (88 g, 0.28 mol) in 450 mL of 1:2 dry THF/toluene under Ar was slowly added 2.5 M n-BuLi (136 mL, 0.34 mol) in hexane at a rate that maintained the temperature below −55°. After stirring for 10 minutes following the addition, this solution was transferred by cannula to a stirred −78° solution of Part C 2,3,4,6-tetra-O-trimethylsilyl-D-glucolactone (153 g, 0.33 mol) in toluene (350 mL) at a rate that maintained the reaction below −55°. The solution was stirred for 30 min at −78° prior to quenching by addition of 400 mL of MeOH containing methanesulfonic acid (28 mL, 0.45 mol). The reaction was stirred overnight for 18 hr at 20° C. The reaction was stirred overnight for 18 hr at 20° C. HPLC analysis revealed a new peak which by LC/MS correspond to the mass of the expected O-methylglucoside. The reaction, once complete, was quenched by the addition of NaHCO₃ (42 g, 0.5 mol) in 200 mL of H₂O. If the pH was not weakly basic, more NaHCO₃ was added prior to dilution 2 fold with H₂O and 3 extractions with EtOAc. The combined EtOAc fractions were washed with brine and dried over Na₂SO₄. After concentration using a rotary evaporator, the oil (140 g, 90% pure by HPLC analysis) was not further purified but instead was carried forward as an impure diastereomeric mixture.

¹H NMR (400 MHz, CDCl₃) δ 7.37 (m, 1H), 7.23 (m, 2H), 7.02 (m, 4H), 5.14 (m, 1H), 5.06 (m, 1H), 4.07 (m, 1H), 4.03 (d, 1H, J_{AB}=15.4 Hz), 3.97 (d, 1H, J_{AB}=15.4 Hz), 3.80-3.70 (m, 4H), 3.60 (m, 1H), 3.48 (m, 1H), 3.31 (m, 1H), 2.84 (s, 3H), 2.53 (q, 2H, J=7.5 Hz), 1.14 (t, 3H, J=7.5 Hz).

¹³C NMR (100 MHz, CDCl₃) δ 144.4, 140.7, 138.94, 136.9, 132.51, 131.6, 130.96, 130.6, 130.2, 129.16, 103.36, 77.0, 74.86, 72.48, 64.27, 51.57, 41.33, 30.75, 17.9.

HPLC retention time: 4.28 min, 90% pure, YMC S5 C-18 4.6×50 mm column, 2.5 mL/min, detection at 220 nM; 4 min gradient 0-100% B hold 2 min at 100% B. Solvent A: 10% MeOH/H₂O+0.2% H₃PO₄. Solvent B: 90% MeOH/H₂O+0.2% H₃PO₄.

LC/MS: [M-OMe]⁺391, 393; [M+Na]⁺445, 447.

### E.

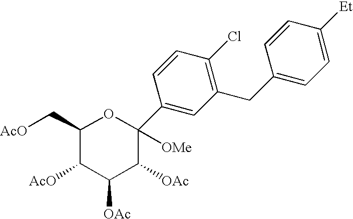

A solution of Part D O-methylglucoside (206 g, 0.49 mol) in THF (1 L) containing diisopropylethylamine (465 g, 3.6 mol) and DMAP (0.5 g, 4.1 mmol) was cooled to 0° C. Acetic anhydride (326 g, 3.19 mol) was slowly added at such a rate that the temperature did not exceed 5° C. After the solution gradually warmed to 20° C., it was stirred for 10 hours whereupon tlc analysis revealed complete conversion to the tetraacetate. The reaction was quenched by addition of EtOAc (1.5 L) and 10% aq. H₃PO₄ (1.5 L). After separation of the layers, the aq. phase was extracted 2× with EtOAc. The combined organic phases were washed 1× with brine prior to drying over Na₂SO₄ and concentration under vacuum. The resultant oil was dissolved twice in 300 mL of toluene and reconcentrated to yield a thick oil (300 g, 95% HPLC purity) that was used without further purification of the resulting impure diastereomeric mixture.

¹H NMR (400 MHz, CDCl₃) δ 7.38 (d, 1H, J=8.3 Hz), 7.28 (dd, 1H, J=8.3 Hz, J=2.2 Hz), 7.24 (d, 1H, J=2.2 Hz), 7.11 (d, 2H, J_{AB}=8.3 Hz), 7.04 (d, 2H, J_{AB}=8.3 Hz), 5.56 (t, 1H, J=9.7 Hz), 5.21 (t, 1H, J=10.1 Hz), 4.93 (t, 1H, J=10.1 Hz), 4.20 (dd, 1H, J=12 Hz, J=2 Hz), 4.12 (d, 1H, J_{AB}=15.4 Hz), 4.02 (m, 1H), 4.018 (d, 1H, J_{AB}=15.4 Hz), 3.10 (s, 3H), 2.606 (q, 2H, J=7.7 Hz), 2.097 (s, 3H), 2.05 (s, 3H), 1.94 (s, 3H), 1.72sd (s, 3H), 1.21 (t, 3H, J=7.7 Hz).

¹³C NMR (100 MHz, CDCl₃) δ 170.7, 170.05, 169.47, 168.9, 142.2, 138.74, 136.4, 135.1, 134.7, 129.8, 129.4, 128.6, 128.0, 126.0, 100.02, 73.83, 71.33, 68.87, 68.77, 62.11, 49.43, 38.75, 28.4, 22.64, 20.68, 20.58, 20.16, 15.5.

HPLC retention time: 4.81 min, 90% pure, YMC S5 C-18 4.6×50 mm column, 2.5 mL/min, detection at 220 nM; 4 min gradient 0-100% B hold 2 min at 100% B. Solvent A: 10% MeOH/H₂O+0.2% H₃PO₄. Solvent B: 90% MeOH/H₂O+0.2% H₃PO₄.

### F.

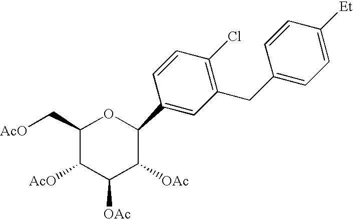

A stirred solution of the above crude oil (301 g, 0.51 mol) in CH₂Cl₂ (500 mL) containing one equivalent of H₂O (9 g, 0.5 mol) and Et₃SiH (188 g, 1.62 mol) was cooled to −20° C. prior to addition of BF₃.Et₂O (145 g, 1.02 mol). During the addition, the temperature was maintained <0° C. The reaction was subsequently stirred 2 hr at 10° C. and 18 hr at 15-20° C. before being quenched by addition of CH₂Cl₂ (500 mL) and H₂O (500 mL). After separation of the layers, the aq phase was extracted once with CH₂Cl₂. The combined organic layers were washed 1× with aq NaHCO₃ and brine prior to drying over Na₂SO₄. After removal of the Na₂SO₄ by filtration, Ac₂O (6.4 g, 65 mmol), diisopropylethylamine (9.5 g, 74 mmol) and DMAP (100 mg, 0.8 mmol) were added. The solution was stirred at 20° C. for 18 hr to insure that any glucoside hydroxyls that hydrolyzed during the reduction and work-up were reacetylated. The oil, obtained after concentration under vacuum, crystallized upon addition of EtOH. After filtration the purity of this material by HPLC was 98%; recrystallization from EtOH yielded the tetraacetylated beta-C-glucoside as a white solid (180 g, 99.8% purity). The overall conversion for procedures D-F was 61%.

HPLC retention time: 4.74 min, 100% pure, YMC S5 C-18 4.6×50 mm column, 2.5 mL/min, detection at 220 nM; 4 min gradient 0-100% B hold 2 min at 100% B. Solvent A: 10% MeOH/H₂O+0.2% H₃PO₄. Solvent B: 90% MeOH/H₂O+0.2% H₃PO₄.

¹H NMR (500 MHz, CDCl₃) δ 7.35 (d, 1H, J=8.2 Hz), 7.19 (dd, 1H, J=8.2 Hz, J=2.2 Hz), 7.11 (d, 2H, J_{AB}=8.5 Hz), 7.086 (d, 1H, J=2.2 Hz), 7.06 (d, 2H, J_{AB}=8.5 Hz), 5.28 (t, 1H, J=9.7 Hz), 5.20 (t, 1H, J=9.7 Hz), 5.04 (t, 1H, J=9.7 Hz), 4.31 (d, 1H, J=9.9 Hz), 4.26 (dd, 1H, J=12 Hz, J=5 Hz), 4.135 (dd, 1H, J=12 Hz, J=5 Hz), 4.095 (d, 1H, J_{AB}=7.7 Hz), 3.995 (d, 1H, J_{AB}=7.7 Hz), 3.79 (m, 1H), 2.605 (q, 2H, J=7.7 Hz), 2.069 (s, 3H), 2.04 (s, 3H), 1.98 (s, 3H), 1.67 (s, 3H), 1.21 (t, 3H, J=7.7 Hz).

¹³C NMR (125 MHz, CDCl₃) δ 170.64, 170.3, 169.4, 168.7, 142.2, 138.78, 136.4, 135.1, 134.6, 129.9, 129.8, 128.7, 128.0, 125.9, 79.45, 76.1, 74.1, 72.5, 68.45, 62.2, 38.6, 28.4, 20.7, 20.6, 20.59, 20.2, 15.55.

LC/MS: [M+NH₄⁺] at m/z 578.3

### Example 2

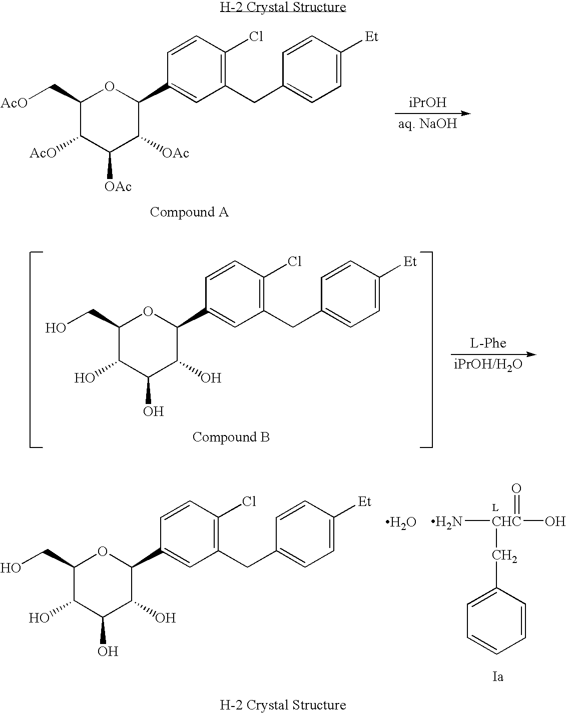

4 g (71.5 mmol) of Compound A was charged into a 250 mL 3-neck flask equipped with mechanical stirrer. 12 mL of isopropylalcohol (IPA) in admixture with 6 ml of 6.3N NaOH was added into the flask and the solution heated to 50° C. After reaction, the solution became clear (˜1 hr). The solution was cooled down to 20° C. and 28 mL of H₂O was charged and the solution stirred for at least 20-30 min. The reaction mixture was neutralized with HCl: 1.1 mL of concentrated HCl (37%) was added to the reaction mixture, pH 7.4; another 50 μL of HCl was added, pH 6.3.

1.18 g of L-Phenylalanine (L-Phe) was charged to the flask. 14 mL of H₂O was used to (1) rinse L-Phe on the flask wall into reaction solution and (2) to adjust to solvent composition to approx. 20 vol % of IPA. The slurry was heated to 70° C. and a clear solution was obtained. The solution was cooled to approx. 50° C. and seeds of form H-2 (Ic) were introduced as slurry (0.5% seeds, 20 mg/200 mL 15% IPA/H₂O solution); The solution turned cloudy immediately. The slurry was cooled to 40° C. over 2 hrs and the temperature maintained for 6 hrs; then the slurry was cooled to 20° C. over 2 hrs and the temperature maintained for approx. 8 hr. The slurry was sampled: a small amount of slurry was centrifuged to isolate solid. The slurry was filtered using a Buchner funnel and filter paper (Whatman 4). The cake was washed with H₂O (4×4 mL); then the cake was washed with EtOAc (3×4 mL). The cake was dried under house vacuum at ˜30° C. overnight to yield compound Ic in the form of H-2 crystals (81% yield).

Note, IPA/H₂O ratio is critical for control of the crystal structure. If IPA is more than 30%, the mixture tends to oil out with time. If IPA is less than 12%, the mixture remains to be a slurry even at 80° C., which makes it difficult to control the crystallization process. To control this critical parameter, it is important to (1) operate in a closed vessel, and (2) monitor the addition of IPA and H₂O, including that from NaOH and HCl.

The seeds of compound Ic are prepared employing the procedure set out as in the Example 2 reaction scheme without the use of seeding.

### Example 3

### H-1 Crystal Structure

Using the process described above for Example 2 for H-2, lesser amounts of crystals of H-1 are formed. Form H-1 is a different form of the same 1:1 L-phenylalanine structure. The H-1 form may be isolated and characterized using procedures known to those skilled in the art, such as described herein.

### Example 4

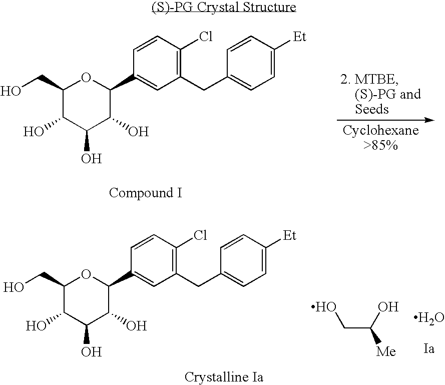

Compound I (1.5 gm, 3.8 mmol) was dissolved in MTBE (8 mL); the solution was slightly cloudy. To this was added (S)-1,2-propane diol[(S)—PG] (397 mg, 2 mL in MTBE). The reaction was stirred for five minutes; no solid was observed. Optionally, it is preferred, but not entirely necessary to add seed crystals (5 mg), at this stage, as is commonly known to one skilled in the art. Immediately cloudiness appeared and crystallization was observed. The reaction was stirred for 24 hr at room temperature, then filtered, and solid obtained was further dried in a desiccator at RT. A total of 1.10 gm complex was isolated. Mother liquor was cooled and an additional 0.5 gm complex was produced after drying. A total 1.55 gm (83%) product was isolated.

The seed crystals employed may be prepared by dissolving compound I in MTBE and treating the resulting solution with (S)-propylene glycol and proceeding as described above (without seeding) to form crystalline compound Ia.

### Crystal Characterization

Crystal structures equivalent to the crystal structures described below and claimed herein may demonstrate similar, yet non-identical, analytical characteristics within a reasonable range of error, depending on test conditions, purity, equipment and other common variables known to those skilled in the art.

Accordingly, it will be apparent to those skilled in the art that various modifications and variations can be made in the present invention without departing from the scope and sprit of the invention. Other embodiments of the invention will be apparent to those skilled in the art from consideration of the specification and practice of the invention disclosed herein. Applicants intend that the specification and examples be considered as exemplary, but not limiting in scope.

### X-Ray Powder Diffraction

One of ordinary skill in the art will appreciate that a powder X-ray diffraction pattern may be obtained with a measurement error that is dependent upon the measurement conditions employed. In particular, it is generally known that intensities in a X-ray powder diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional powder X-ray powder diffraction pattern is typically about 5% or less, and such degree of measurement error should be taken into account as pertaining to the aforementioned diffraction angles. Consequently, it is to be understood that the crystal structures of the instant invention are not limited to the crystal structures that provide X-ray diffraction patterns completely identical to the X-ray powder diffraction patterns depicted in the accompanying Figures disclosed herein. Any crystal structures that provide powder X- ray diffraction patterns substantially identical to those disclosed in the accompanying Figures fall within the scope of the present invention. The ability to ascertain substantial identities of X-ray powder diffraction patterns is within the purview of one of ordinary skill in the art.

### H-2 Structure

About 200 mg of compound Ic (prepared as described in Example 2) were pack into a Philips powder X-ray diffraction (PXRD) sample holder. The sample was transferred to a Philips MPD unit (45 KV, 40 mA, Cu Kα₁). Data were collected at room temperature in the 2 to 32 2-theta rage (continuous scanning mode, scanning rate 0.03 degrees/sec., auto divergence and anti scatter slits, receiving slit: 0.2 mm, sample spinner: ON)

### (S)—PG Structure

X-ray powder diffraction (PXRD) data were obtained using a Bruker C2 GADDS. The radiation was Cu Kα (40 KV, 50 mA). The sample-detector distance was 15 cm. Powder samples of the (S)—PG compound Ia (prepared as described in Example 3) were placed in sealed glass capillaries of 1 mm or less in diameter; the capillary was rotated during data collection. Data were collected for 3≦2θ≦35° with a sample exposure time of at least 2000 seconds. The resulting two-dimensional diffraction arcs were integrated to create a traditional 1-dimensional PXRD pattern with a step size of 0.02 degrees 2Θ in the range of 3 to 35 degrees 2Θ.

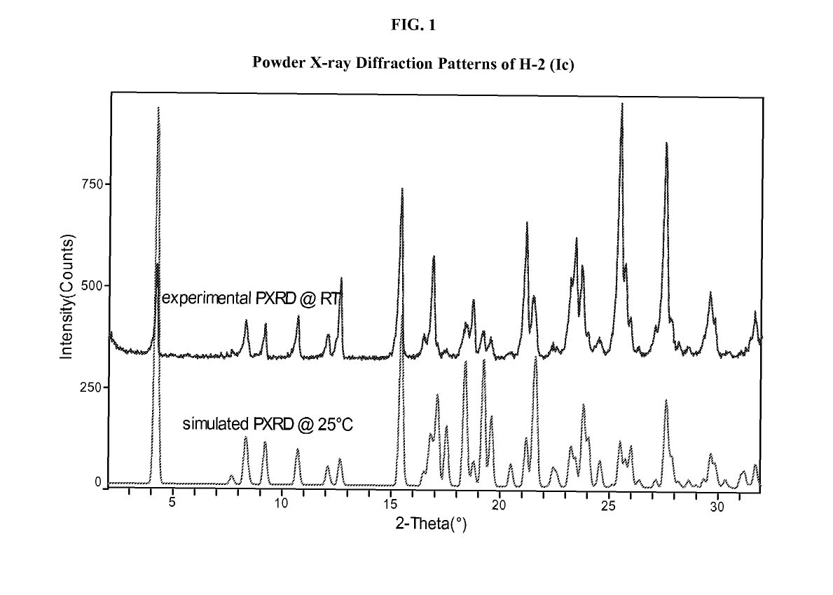

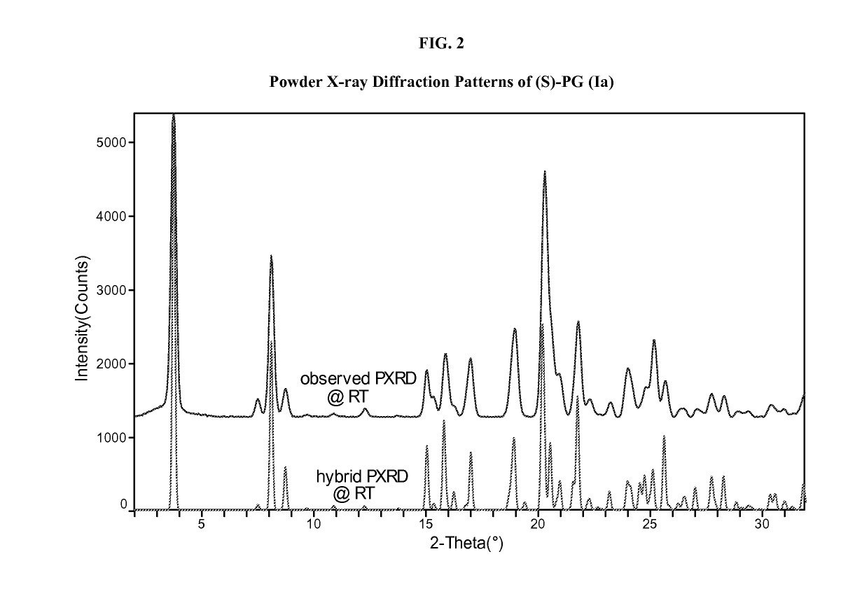

Powder X-ray diffraction patterns for the Ic H-2 and Ia (S)—PG structures are illustrated in FIGS. 1-2, respectively. Selected diffraction peak positions (degrees 2Θ±0.2) for each of the structures H-2 and (S)—PG are shown in Table 1 below. Characteristic diffraction peak positions (degrees 2θ±0.1) @ RT, based on a high quality pattern collected with a diffractometer (CuKα) with a spinning capillary with 2θ calibrated with a National Institute of Standards and Technology methodology, other suitable standard known to those skilled in the art. The relative intensities, however, may change depending on the crystal size and morphology.

TABLE 1
Selected PXRD Peaks (2θ ± 0.1°)

| Ic H-2 | Ia (S)-PG |
|---|---|
| 4.2 | 3.7 |
| 8.3 | 8.1 |
| 9.2 | 8.7 |
| 10.7 | 15.0 |
| 15.5 | 15.8 |
| 18.4 | 17.0 |
| 19.2 | 18.9 |
| 21.6 | 20.2 |
| | 21.8 |

Hybrid PXRD Pattern (from Isostructural Analog): (S)—PG

“Hybrid” simulated powder X-ray patterns were generated as described in the literature (Yin, S. et al., American Pharmaceutical Review, 6(2):80 (2003)). The room temperature cell parameters were obtained by performing a cell refinement using the CellRefine.xls program. Input to the program includes the 2-theta position of ca. 10 reflections, obtained from the experimental room temperature powder pattern; the corresponding Miller indices, hkl, were assigned based on the single-crystal data collected for an isostructural analog. A crystal structure for the molecule of interest was generated in a two step process: (1) by replacing the analog molecule in the experimental analog crystal structure with the molecule of interest. This step fixes the orientation and position of the molecule of interest in the unit cell of the analog compound; (2) inserting the molecule of interest into the room temperature cell obtained from the experimental PXRD of the molecule of interest, as described above. In this step, the molecules are inserted in a manner that retains the size and shape of the molecule and the position of the molecules with respect to the cell origin, but, allows intermolecular distances to expand/contract with the cell. A new (hybrid) PXRD was calculated (by either of the software programs, Alex or LatticeView) based on the crystal structure generated as described above.

Cell parameters for single crystal (input at 25°) from analog and hybrid (refined at RT) of (S)—PG complex Ia are in Table 2 below. Crystal structures demonstrating substantially similar input and refined cell parameters, are deemed to fall within the scope of the invention (i.e., equivalent to the (S)—PG Ia data illustrated below).

TABLE 2
(S)-PG Ia

| Cell Parameter | Input | Refined |
|---|---|---|
| a (Å) | 11.26 | 11.219 |
| b (Å) | 4.809 | 4.782 |
| c (Å) | 46.723 | 47.124 |
| α (°) | 90 | 90 |
| β (°) | 90 | 90 |
| γ (°) | 90 | 90 |
| V (Å³) | 2532.15 | 2528.17 |

V is the volume of the unit cell.

### Solid-State Nuclear Magnetic Resonance

Structure (S)—PG Ia was characterized by solid state NMR techniques.

All solid-state C-13 NMR measurements were made with a Bruker DSX-400, 400 MHz NMR spectrometer. High resolution spectra were obtained using high-power proton decoupling and the TPPM pulse sequence and ramp amplitude cross-polarization (RAMP-CP) with magic-angle spinning (MAS) at approximately 12 kHz (Bennett, A. E. et al., J. Chem. Phys., 103:6951 (1995)), (Metz, G. et al., J. Magn. Reson. A, 110:219-227 (1994)). Approximately 70 mg of sample, packed into a canister-design zirconia rotor was used for each experiment. Chemical shifts (6) were referenced to external adamantane with the high frequency resonance being set to 38.56 ppm (Earl, W. L. et al., J. Magn. Reson., 48:35-54 (1982)).

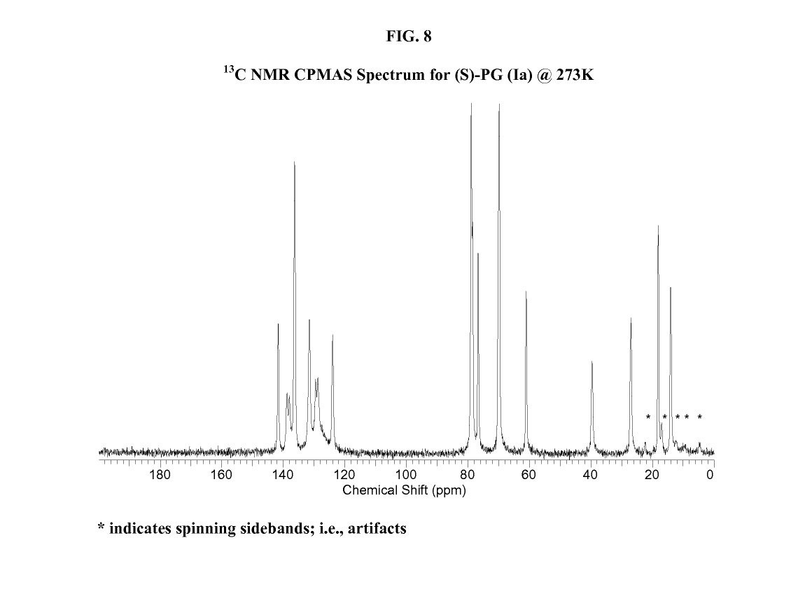

The resulting ¹³C NMR CPMAS spectrum for structure (S)—PG Ia is shown in FIG. 8.

The major resonance peaks for the solid state carbon spectrum of the (S)—PG Ia structure is listed below in Table 3. Crystal structures demonstrating substantially similar ¹³C NMR peak positions, wherein “substantially similar” means 10 to 15% of dimensionless value, are deemed to fall within the scope of the invention (i.e., equivalent to the (S)—PG Ia structure illustrated below).

TABLE 3
(S)-PG Ia
SSNMR Peak Positions/δ (in ppm) Relative to TMS (Tetramethyl Silane)
Determined at 273° K
δ/ppm

14.1
18.1
27.0
39.6
61.1
69.9
76.7
78.5
78.9
124.0
128.8*
129.5*
131.5
136.3
138.1*
138.9*
141.7

These data are strictly valid for a 400 MHz spectrophotometer.

### Thermal Gravimetric Analysis

Thermal gravimetric analysis (TGA) experiments were performed in a TA Instruments™ model Q500. The sample (about 10-30 mg) was placed in a platinum pan previously tared. The weight of the sample was measured accurately and recorded to a thousand of a milligram by the instrument The furnace was purged with nitrogen gas at 100 mL/min. Data were collected between room temperature and 300° C. at 10° C./min heating rate.

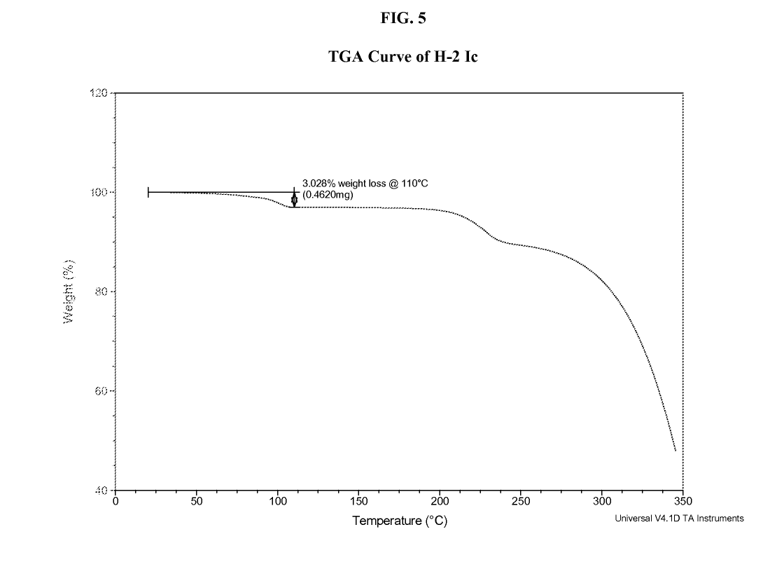

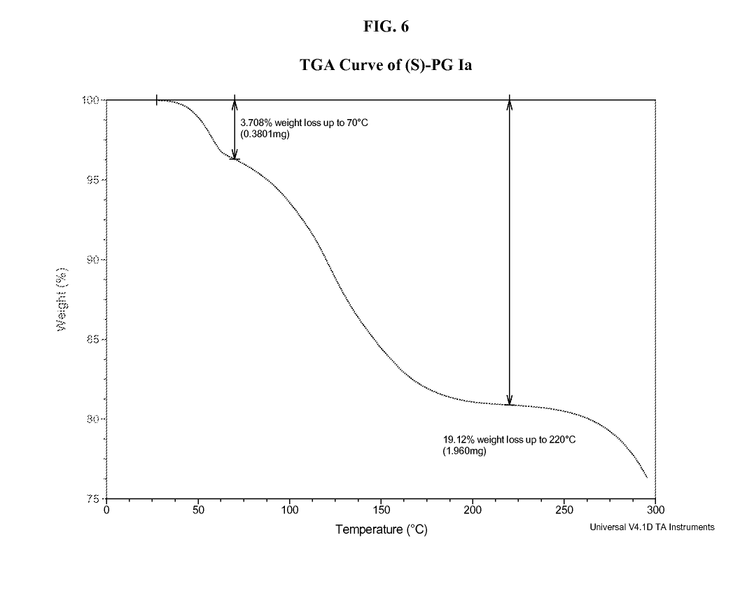

TGA curves for structures H-2 Ic and (S)—PG Ia are shown in FIGS. 5 and 6 respectively. Weight loss corresponds to one mole of water per mole of structure analyzed. Equivalent crystal structures may demonstrate similar weight loss within a reasonable range as illustrated in FIGS. 5 and 6, depending on testing conditions, purity and other variables known to those skilled in the art.

### Differential Scanning Calorimetry

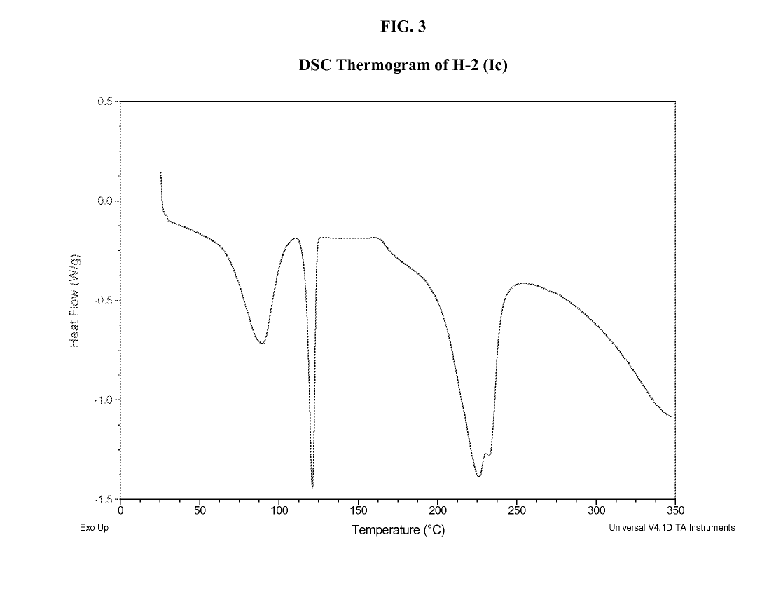

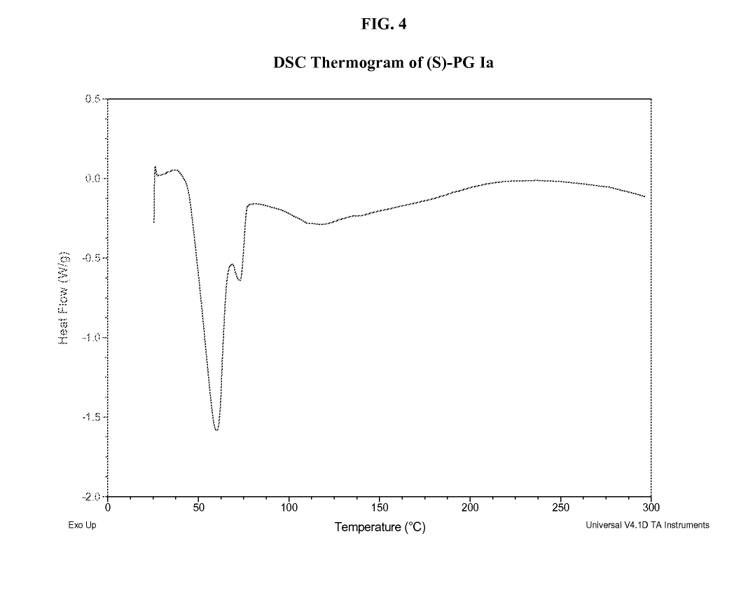

The solid state thermal behavior of structures H-2 Ic and (S)—PG Ia were investigated by differential scanning calorimetry (DSC). The DSC curves for structures H-2 Ic and (S)—PG Ia are shown in FIGS. 3 and 4 respectively.

Differential scanning calorimetry (DSC) experiments were performed in a TA Instruments™ model Q1000. The sample (about 2-6 mg) was weighed in an aluminum pan and recorded accurately to a hundredth of a milligram, and transferred to the DSC. The instrument was purged with nitrogen gas at 50 mL/min. Data were collected between room temperature and 300° C. at 10° C./min heating rate. The plot was made with the endothermic peaks pointing down.

One of ordinary skill in the art will, however, note that in DSC measurement there is a certain degree of variability in actual measured onset and peak temperatures, depending on rate of heating, crystal shape and purity, and other measurement parameters.

### Moisture Sorption Isotherms

Moisture sorption isotherms were collected in a VTI SGA-100 Symmetric Vapor Analyzer using approximately 10 mg of sample. The sample was dried at 60° C. until the loss rate of 0.0005 wt %/min was obtained for 10 minutes. The sample was tested at 25° C. and 3 or 4, 5, 15, 25, 35, 45, 50, 65, 75, 85, and 95% RH. Equilibration at each RH was reached when the rate of 0.0003 wt %/min for 35 minutes was achieved or a maximum of 600 minutes.

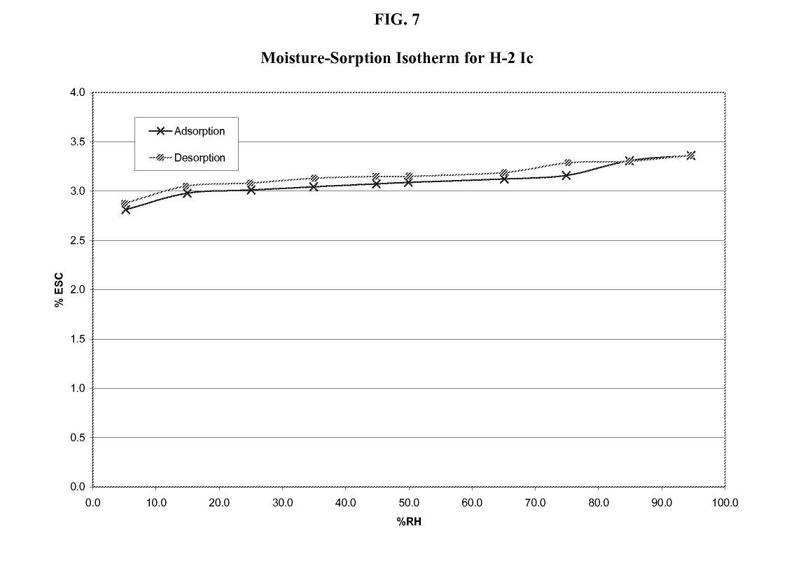

Moisture sorption isotherms for the H-2 Ic structure is shown in FIG. 7.

### Single Crystal X-Ray Analysis

A single crystal analyses for the H-1, H-2 and (S)—PG structures were obtained and investigated by x-ray diffraction.

Data were collected on a Bruker-Nonius¹ CAD4 serial diffractometer. Unit cell parameters were obtained through least-squares analysis of the experimental diffractometer settings of 25 high-angle reflections. Intensities were measured using Cu Kα radiation (λ=1.5418 Å) at a constant temperature with the θ-2θ variable scan technique and were corrected only for Lorentz-polarization factors. Background counts were collected at the extremes of the scan for half of the time of the scan. Alternately, single crystal data were collected on a Bruker-Nonius Kappa CCD 2000 system using Cu Kα radiation (λ=1.5418 Å). Indexing and processing of the measured intensity data were carried out with the HKL2000 software package² in the Collect program suite.³

When indicated, crystals were cooled in the cold stream of an Oxford cryo system⁴ during data collection. ¹BRUKER AXS, Inc., 5465 East Cheryl Parkway Madison, Wis. 53711 USA.²Otwinowski, Z. & Minor, W. in Macromolecular Crystallography, Academic, NY, publ., Carter, W. C. Jr., & Sweet, R. M., eds. Vol.276, pp.307-326 (1997).³Collect Data collection and processing user interface: Collect: Data collection software, R. IIooft, Nonius B. V., 1998.⁴Oxford Cryosystems Cryostream cooler: Cosier, J. et al., J. Appl. Cryst., 19:105 (1986).

The structures were solved by direct methods and refined on the basis of observed reflections using either the SDP⁵software package with minor local modifications or the crystallographic package, MAXUS.⁶

The derived atomic parameters (coordinates and temperature factors) were refined through full matrix least-squares. The function minimized in the refinements was Σ_{W}(|F_{O}|−|F_{C}|)². R is defined as Σ∥F_{O}|−|F_{C}∥/Σ|F_{O}| while R_{W}=[Σ_{W}(|F_{O}|−|F_{C}|)²/Σ_{W}|F_{O}|²]^{1/2} where w is an appropriate weighting function based on errors in the observed intensities. Difference maps were examined at all stages of refinement. Hydrogens were introduced in idealized positions with isotropic temperature factors, but no hydrogen parameters were varied.

Unit cell parameters for the H-2 Ic, H-1 Ib and (S)—PG Ia structures are listed below in Table 4. As used herein, the unit cell parameter “molecules per cell” refers to the number of molecules of Compound in the unit cell.

TABLE 4
Crystallographic Unit Cell Data

| Structure | T | a(Å) | b (Å) | c (Å) | α^{°} | β^{°} | γ^{°} | Vₘ | Z′ | sg | Dcalc | R |
|---|---|---|---|---|---|---|---|---|---|---|---|---|
| H-2 | 25 | 11.564 (4)) | 5.954 (1) | 21.123 (6) | — | 96.96 (2) | — | 722 | 1 | P2₁ | 1.325 | 0.09 |
| H-1 | 22 | 11.738 (3) | 5.882 (1) | 21.001 (4) | — | 86.66 (1) | — | 724 | 1 | P2₁ | 1.322 | 0.06 |
| (S)-PG | RT | 11.269 | 4.809 | 46.723 | 90 | 90 | 90 | 633 | 1 | P2₁2₁2₁ | 1.280 | n/a |

T = temp (° C.) for the crystallographic data.
Z′ = number of drug molecules per asymmetric unit.
Vₘ = V(unit cell)/(Z drug molecules per cell).
R = residual index (I > 3sigma(I)).
D_{calc} = density of crystal calculated.
SG = space group

Numerical values illustrated within brackets ( ) denote estimated standard deviations in least significant figures.

Table 5 below sets forth the positional parameters and their estimated standard deviations for the H-2 Ic structure at 25° C.

TABLE 5
Fractional Atomic Coordinates of H-2 Ic at T = 25° C.

| Atom | X | Y | Z |
|---|---|---|---|
| O2 | 0.425790 | 0.326243 | 0.095241 |
| O3 | 0.360534 | 0.048059 | 0.197261 |
| O4 | 0.817546 | −0.376218 | 0.110602 |
| O5 | 0.575357 | 0.107858 | 0.015826 |
| O6 | 0.642678 | −0.183012 | 0.168918 |
| O28 | 0.161491 | 0.048951 | 0.110224 |
| O31 | 0.018684 | 0.206760 | 0.046833 |
| O40 | 0.233292 | −0.164897 | 0.001652 |
| N30 | 0.057585 | 0.626076 | 0.085720 |
| CL1 | 0.556701 | −0.477228 | 0.462238 |
| C7 | 0.476664 | 0.045625 | 0.175701 |
| C8 | 0.587598 | 0.083175 | 0.083721 |
| C9 | 0.526923 | −0.185223 | 0.187324 |
| C10 | 0.462457 | 0.093957 | 0.103479 |
| C11 | 0.611333 | −0.192925 | 0.368809 |
| C12 | 0.768170 | −0.167025 | 0.089052 |
| C13 | 0.539544 | −0.246761 | 0.256622 |
| C14 | 0.639478 | −0.154381 | 0.101149 |
| C15 | 0.553521 | −0.385669 | 0.383484 |
| C16 | 0.600311 | −0.135519 | 0.304486 |
| C17 | 0.813157 | −0.071579 | 0.417965 |
| C18 | 0.486279 | −0.502564 | 0.338024 |
| C19 | 0.684371 | −0.052855 | 0.418487 |
| C20 | 1.057704 | −0.136226 | 0.410352 |
| C21 | 0.992178 | −0.294378 | 0.435034 |
| C22 | 0.880056 | 0.087791 | 0.393559 |
| C23 | 0.477265 | −0.443409 | 0.273057 |
| C24 | 0.997174 | 0.059657 | 0.390805 |
| C25 | 0.871064 | −0.276155 | 0.440042 |
| C26 | 1.186347 | −0.180502 | 0.407455 |
| C27 | 1.244817 | −0.017583 | 0.373440 |
| C29 | −0.029273 | 0.254132 | 0.208117 |
| C32 | 0.141162 | 0.452919 | 0.115046 |
| C33 | 0.149794 | 0.459646 | 0.188348 |
| C34 | 0.037491 | 0.452935 | 0.216726 |
| C35 | −0.186729 | 0.442558 | 0.259630 |
| C36 | 0.106575 | 0.213986 | 0.087732 |
| C37 | −0.005529 | 0.636616 | 0.246231 |
| C38 | −0.140156 | 0.252341 | 0.229007 |
| C39 | −0.114504 | 0.628622 | 0.268705 |
| H21 | 0.442900 | 0.374500 | 0.051100 |
| H31 | 0.300200 | 0.037600 | 0.157600 |
| H41 | 0.801400 | −0.493500 | 0.075800 |
| H51 | 0.644800 | 0.185700 | 0.002100 |
| H301 | 0.045000 | 0.609000 | 0.038100 |
| H302 | −0.022200 | 0.598500 | 0.101700 |
| H71 | 0.531200 | 0.166300 | 0.205000 |
| H81 | 0.636800 | 0.228000 | 0.105100 |
| H91 | 0.471400 | −0.307800 | 0.156600 |
| H101 | 0.390800 | −0.017200 | 0.080400 |
| H121 | 0.811600 | −0.021100 | 0.111400 |
| H122 | 0.771600 | −0.141700 | 0.035600 |
| H141 | 0.577700 | −0.287300 | 0.075700 |
| H161 | 0.647600 | 0.015800 | 0.291800 |
| H181 | 0.433800 | −0.638400 | 0.353000 |
| H191 | 0.657000 | 0.125800 | 0.408000 |
| H192 | 0.658000 | −0.086900 | 0.466200 |
| H211 | 1.038400 | −0.452300 | 0.454500 |
| H221 | 0.839400 | 0.252500 | 0.377000 |
| H231 | 0.421900 | −0.548900 | 0.237100 |
| H241 | 1.047100 | 0.193800 | 0.371300 |
| H251 | 0.825600 | −0.417600 | 0.461100 |
| H261 | 1.233100 | −0.200900 | 0.455500 |
| H262 | 1.196500 | −0.344000 | 0.382700 |
| H271 | 1.339100 | −0.051000 | 0.372500 |
| H272 | 1.206400 | 0.008000 | 0.325000 |
| H273 | 1.243000 | 0.151100 | 0.397800 |
| H291 | 0.006900 | 0.100600 | 0.186100 |
| H321 | 0.225200 | 0.496800 | 0.099600 |
| H331 | 0.203100 | 0.314500 | 0.207300 |
| H332 | 0.199000 | 0.610600 | 0.206700 |
| H351 | −0.275600 | 0.447800 | 0.272000 |
| H371 | 0.047400 | 0.797800 | 0.251300 |
| H381 | −0.194200 | 0.103400 | 0.222300 |
| H391 | −0.139900 | 0.766400 | 0.298100 |
| H303 | 0.082200 | 0.780500 | 0.097400 |
| H401 | 0.207200 | −0.097600 | 0.040600 |
| H402 | 0.160200 | −0.247800 | −0.021800 |

Table 6 below sets forth the positional parameters and therein estimated standard deviations for the H-1 Ib structure at 25° C.

TABLE 6
Table of Fractional Atomic Coordinates for From H-1 Ib at T = 22° C.

| Atom | X | Y | Z |
|---|---|---|---|
| CL1 | 0.426751 | −0.500300 | 0.457418 |
| O2 | 0.589162 | −0.169159 | 0.168185 |
| O3 | 0.387602 | 0.324987 | 0.092328 |
| O4 | 0.575306 | 0.12412 | 0.015007 |
| C5 | 0.558088 | 0.096139 | 0.082309 |
| O6 | 0.294746 | 0.029018 | 0.190498 |
| C7 | 0.413687 | 0.041088 | 0.172603 |
| O8 | 0.784217 | −0.348975 | 0.111208 |
| C9 | 0.472310 | −0.192807 | 0.186673 |
| O10 | 0.232785 | −0.147957 | 0.001140 |
| O11 | 0.125306 | 0.078328 | 0.108142 |
| N12 | 0.028163 | 0.648639 | 0.086383 |
| C13 | −0.028752 | 0.476730 | 0.217888 |
| O14 | 0.000245 | 0.232300 | 0.045029 |
| C15 | 0.608531 | −0.130069 | 0.099798 |
| C16 | 0.387325 | −0.448891 | 0.273359 |
| C17 | 0.171988 | −0.686670 | 0.367141 |
| C18 | 0.091560 | 0.483651 | 0.188943 |
| C20 | 0.373705 | −0.514950 | 0.335568 |
| C21 | 0.736134 | −0.135773 | 0.089100 |
| C22 | 0.103948 | 0.468262 | 0.115500 |
| C23 | −0.084673 | 0.659260 | 0.250634 |
| C24 | 0.436000 | −0.402433 | 0.378303 |
| C25 | 0.453020 | −0.267014 | 0.254464 |
| C26 | −0.254127 | 0.443123 | 0.263222 |
| C27 | 0.301284 | −0.726389 | 0.348956 |
| C28 | 0.503219 | −0.219896 | 0.362346 |
| C29 | −0.095222 | 0.288655 | 0.208609 |
| C30 | −0.194869 | 0.640701 | 0.274492 |
| C31 | −0.203431 | 0.263479 | 0.233000 |
| C32 | 0.027966 | −0.461534 | 0.422895 |
| C33 | 0.067777 | 0.237032 | 0.084630 |
| C34 | 0.510998 | −0.149400 | 0.299164 |
| C35 | 0.133448 | −0.487385 | 0.398389 |
| C36 | 0.097978 | −0.854982 | 0.358084 |
| C37 | −0.012548 | −0.833011 | 0.380679 |
| C38 | −0.184225 | −0.642273 | 0.433591 |
| C39 | −0.050911 | −0.634923 | 0.413778 |
| C40 | −0.221079 | −0.458194 | 0.463434 |
| H41 | 0.604600 | 0.239400 | 0.107500 |
| H42 | 0.387500 | −0.014600 | 0.071900 |
| H43 | 0.567700 | −0.267300 | 0.073400 |
| H44 | 0.771100 | 0.014000 | 0.115100 |
| H45 | 0.761300 | −0.102600 | 0.038200 |
| H46 | 0.452600 | 0.180800 | 0.201400 |
| H47 | 0.434000 | −0.314000 | 0.157000 |
| H48 | 0.337200 | −0.528600 | 0.234100 |
| H49 | 0.560900 | 0.011700 | 0.282200 |
| H50 | 0.552500 | −0.119600 | 0.399200 |
| H51 | 0.338400 | −0.813500 | 0.389000 |
| H52 | 0.309600 | −0.835800 | 0.307700 |
| H53 | 0.128500 | −0.998300 | 0.330300 |
| H54 | 0.195500 | −0.346300 | 0.404900 |
| H55 | 0.004500 | −0.294500 | 0.451400 |
| H56 | −0.075900 | −0.967600 | 0.375200 |
| H57 | −0.230700 | −0.663100 | 0.391200 |
| H58 | −0.200400 | −0.776700 | 0.466100 |
| H59 | −0.310100 | −0.453700 | 0.474700 |
| H60 | −0.173700 | −0.416200 | 0.504200 |
| H61 | −0.204000 | −0.302600 | 0.429300 |
| H62 | −0.343100 | 0.457300 | 0.282900 |
| H63 | −0.257100 | 0.120000 | 0.229400 |
| H64 | −0.233600 | 0.796900 | 0.299200 |
| H65 | −0.058400 | 0.146500 | 0.179900 |
| H66 | −0.030300 | 0.820800 | 0.257300 |
| H67 | 0.133800 | 0.654400 | 0.204300 |
| H68 | 0.143700 | 0.355700 | 0.209100 |
| H69 | 0.195100 | 0.515700 | 0.098600 |
| H70 | 0.035300 | 0.646800 | 0.035700 |
| H71 | −0.059000 | 0.621100 | 0.103800 |
| H72 | 0.051300 | 0.824800 | 0.102100 |
| H73 | 0.220200 | −0.042100 | 0.045800 |
| H74 | 0.148900 | −0.179500 | −0.015000 |
| H75 | 0.246700 | 0.062600 | 0.148300 |
| H76 | 0.420700 | 0.391600 | 0.045000 |
| H77 | 0.653200 | 0.226900 | 0.003800 |
| H78 | 0.783100 | −0.466500 | 0.072900 |

### Utilities and Combinations

### A. Utilities

The compounds of the present invention (S)—PG Ia, H-1 Ib and H-2 Ic possesses activity as an inhibitor of the sodium dependent glucose transporters found in the intestine and kidney of mammals. Preferably, the compound of the invention is a selective inhibitor of renal SGLT2 activity, and therefore may be used in the treatment of diseases or disorders associated with SGLT2 activity.

Accordingly, the compound of the present invention can be administered to mammals, preferably humans, for the treatment of a variety of conditions and disorders, including, but not limited to, treating or delaying the progression or onset of diabetes (including Type I and Type II, impaired glucose tolerance, insulin resistance, and diabetic complications, such as nephropathy, retinopathy, neuropathy and cataracts), hyperglycemia, hyperinsulinemia, hypercholesterolemia, elevated blood levels of free fatty acids or glycerol, hyperlipidemia, dyslipidemia, hypertriglyceridemia, obesity, wound healing, tissue ischemia, atherosclerosis and hypertension. The compounds of the present invention may also be utilized to increase the blood levels of high density lipoprotein (HDL).

In addition, the conditions, diseases, and maladies collectively referred to as “Syndrome X” or Metabolic Syndrome as detailed in Johannsson, J. Clin. Endocrinol. Metab., 82:727-734 (1997), may be treated employing the compound of the present invention.

The crystalline compounds H-1 Ib, H-2 Ic and (S)—PG Ia may be administered in dosage forms and in dosages as disclosed in U.S. application Ser. No. 11/233,617, filed Sep. 23, 2005, the disclosure of which in its entirety is incorporated herein by reference.

### B. Combinations

The present invention includes within its scope pharmaceutical compositions comprising, as an active ingredient, a therapeutically effective amount of a compound of formula I, alone or in combination with a pharmaceutical carrier or diluent. Optionally, the compound of the present invention can be utilized as an individual treatment, or utilized in combination with one or more other therapeutic agent(s).

Other “therapeutic agent(s)” suitable for combination with the compound of the present invention include, but are not limited to, known therapeutic agents useful in the treatment of the aforementioned disorders including: anti-diabetic agents; anti-hyperglycemic agents; hypolipidemic/lipid lowering agents; anti-obesity agents; anti-hypertensive agents and appetite suppressants.

Examples of suitable anti-diabetic agents for use in combination with the compound of the present invention include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, gliclazide, chlorpropamide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, glycogen phosphorylase inhibitors, inhibitors of fatty acid binding protein (aP2), glucagon-like peptide-1 (GLP-1) or other agonists of the GLP-1 receptor, and dipeptidyl peptidase IV (DPP4) inhibitors.

It is believed that the use of the compound of formula I in combination with at least one or more other antidiabetic agent(s) provides antihyperglycemic results greater than that possible from each of these medicaments alone and greater than the combined additive anti-hyperglycemic effects produced by these medicaments.

Other suitable thiazolidinediones include Mitsubishi's MCC-555 (disclosed in U.S. Pat. No. 5,594,016), Glaxo-Wellcome's GL-262570, englitazone (CP-68722, Pfizer) or darglitazone (CP-86325, Pfizer, isaglitazone (MIT/J&J), JTT-501 (JPNT/P&U), L-895645 (Merck), R-119702 (Sankyo/WL), NN-2344 (Dr. Reddy/NN), or YM-440 (Yamanouchi).

Examples of PPAR-alpha agonists, PPAR-gamma agonists and PPAR alpha/gamma dual agonists include muraglitazar, peliglitazar, AR-HO39242 (Astra/Zeneca), GW-409544 (Glaxo-Wellcome), GW-501516 (Glaxo-Wellcome), KRP297 (Kyorin Merck) as well as those disclosed by Murakami et al, “A Novel Insulin Sensitizer Acts As a Coligand for Peroxisome Proliferation—Activated Receptor Alpha (PPAR alpha) and PPAR gamma. Effect on PPAR alpha Activation on Abnormal Lipid Metabolism in Liver of Zucker Fatty Rats”, Diabetes, 47:1841-1847 (1998), WO 01/21602 and in U.S. Pat. No. 6,653,314, the disclosure of which is incorporated herein by reference, employing dosages as set out therein, which compounds designated as preferred are preferred for use herein.

Suitable aP2 inhibitors include those disclosed in U.S. application Ser. No. 09/391,053, filed Sep. 7, 1999, and in U.S. application Ser. No. 09/519,079, filed Mar. 6, 2000, employing dosages as set out herein.

Suitable DPP4 inhibitors include those disclosed in WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) as disclosed by Hughes et al., Biochemistry, 38(36):11597-11603 (1999), TSL-225 (tryptophyl-1,2,3,4-tetrahydroisoquinoline-3-carboxylic acid (disclosed by Yamada et al., Bioorg. & Med. Chem. Lett., 8:1537-1540 (1998)), 2-cyanopyrrolidides and 4-cyanopyrrolidides, as disclosed by Ashworth et al., Bioorg. & Med. Chem. Lett., 6(22):1163-1166 and 2745-2748 (1996), the compounds disclosed in U.S. application Ser. No. 10/899,641, WO 01/68603 and U.S. Pat. No. 6,395,767, employing dosages as set out in the above references.

Other suitable meglitinides include nateglinide (Novartis) or KAD1229 (PF/Kissei).

Examples of suitable anti-hyperglycemic agents for use in combination with the compound of the present invention include glucagon-like peptide-1 (GLP-1,) such as GLP-1(1-36) amide, GLP-1(7-36) amide, GLP-1(7-37) (as disclosed in U.S. Pat. No. 5,614,492), as well as exenatide (Amylin/Lilly), LY-315902 (Lilly), MK-0431 (Merck), liraglutide (NovoNordisk), ZP-10 (Zealand Pharmaceuticals A/S), CJC-1131 (Conjuchem Inc), and the compounds disclosed in WO 03/033671.

Examples of suitable hypolipidemic/lipid lowering agents for use in combination with the compound of the present invention include one or more MTP inhibitors, HMG CoA reductase inhibitors, squalene synthetase inhibitors, fibric acid derivatives, ACAT inhibitors, lipoxygenase inhibitors, cholesterol absorption inhibitors, ileal Na⁺/bile acid co-transporter inhibitors, up-regulators of LDL receptor activity, bile acid sequestrants, cholesterol ester transfer protein (e.g., CETP inhibitors, such as CP-529414 (Pfizer) and JTT-705 (Akros Pharma)), PPAR agonists (as described above) and/or nicotinic acid and derivatives thereof.

MTP inhibitors which may be employed as described above include those disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440.

The HMG CoA reductase inhibitors which may be employed in combination with one or more compound of formula I include mevastatin and related compounds, as disclosed in U.S. Pat. No. 3,983,140, lovastatin (mevinolin) and related compounds, as disclosed in U.S. Pat. No. 4,231,938, pravastatin and related compounds, such as disclosed in U.S. Pat. No. 4,346,227, simvastatin and related compounds, as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin, as disclosed in U.S. Pat. Nos. 5,006,530 and 5,177,080, atorvastatin, as disclosed in U.S. Pat. Nos. 4,681,893, 5,273,995, 5,385,929 and 5,686,104, atavastatin (Nissan/Sankyo's nisvastatin (NK-104)), as disclosed in U.S. Pat. No. 5,011,930, visastatin (Shionogi-Astra/Zeneca (ZD-4522)), as disclosed in U.S. Pat. No. 5,260,440, and related statin compounds disclosed in U.S. Pat. No. 5,753,675, pyrazole analogs of mevalonolactone derivatives, as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No. 0142146 A2, and quinoline and pyridine derivatives, as disclosed in U.S. Pat. Nos. 5,506,219 and 5,691,322.

Preferred hypolipidemic agents are pravastatin, lovastatin, simvastatin, atorvastatin, fluvastatin, cerivastatin, atavastatin and ZD-4522.

In addition, phosphinic acid compounds useful in inhibiting HMG CoA reductase, such as those disclosed in GB 2205837, are suitable for use in combination with the compound of the present invention.

The squalene synthetase inhibitors suitable for use herein include, but are not limited to, α-phosphono-sulfonates disclosed in U.S. Pat. No. 5,712,396, those disclosed by Biller et al., J. Med. Chem., 31(10):1869-1871 (1988), including isoprenoid(phosphinyl-methyl)phosphonates, as well as other known squalene synthetase inhibitors, for example, as disclosed in U.S. Pat. Nos. 4,871,721 and 4,924,024 and in Biller, S. A. et al., Current Pharmaceutical Design, 2:1-40 (1996).

In addition, other squalene synthetase inhibitors suitable for use herein include the terpenoid pyrophosphates disclosed by Ortiz de Montellano, P. et al., J. Med. Chem., 20:243-249 (1977), the farnesyl diphosphate analog A and presqualene pyrophosphate (PSQ-PP) analogs as disclosed by Corey et al., J. Am. Chem. Soc., 98:1291-1293 (1976), phosphinylphosphonates reported by McClard, R. W. et al., J. Am. Chem. Soc., 109:5544 (1987) and cyclopropanes reported by Capson, T. L., PhD dissertation, June, 1987, Dept. Med. Chem. U of Utah, Abstract, Table of Contents, pp. 16, 17, 40-43, 48-51, Summary.

The fibric acid derivatives which may be employed in combination the compound of formula I include fenofibrate, gemfibrozil, clofibrate, bezafibrate, ciprofibrate, clinofibrate and the like, probucol, and related compounds, as disclosed in U.S. Pat. No. 3,674,836, probucol and gemfibrozil being preferred, bile acid sequestrants, such as cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives, such as disclosed in U.S. Pat. No. 4,759,923, quaternary amine poly(diallyldimethylammonium chloride) and ionenes, such as disclosed in U.S. Pat. No. 4,027,009, and other known serum cholesterol lowering agents.

The ACAT inhibitor which may be employed in combination the compound of formula I include those disclosed in Drugs of the Future, 24:9-15 (1999) (Avasimibe); Nicolosi et al., “The ACAT inhibitor, C1-1011 is effective in the prevention and regression of aortic fatty streak area in hamsters”, Atherosclerosis (Shannon, Irel.), 137(1):77-85 (1998); Ghiselli, G., “The pharmacological profile of FCE 27677: a novel ACAT inhibitor with potent hypolipidemic activity mediated by selective suppression of the hepatic secretion of ApoB100-containing lipoprotein”, Cardiovasc. Drug Rev., 16(1):16-30 (1998); Smith, C. et al., “RP 73163: a bioavailable alkylsulfinyl-diphenylimidazole ACAT inhibitor”, Bioorg. Med. Chem. Lett., 6(1):47-50 (1996); Krause, B. R. et al., Chapter 6: “ACAT Inhibitors: Physiologic Mechanisms for Hypolipidemic and Anti-Atherosclerotic Activities in Experimental Animals”, Inflammation: Mediators and Pathways, CRC Press, Inc., publ., Ruffolo, Jr., R. R. et al., eds., pp. 173-198 (1995); Sliskovic et al., “ACAT inhibitors: potential anti-atherosclerotic agents”, Curr. Med. Chem., 1(3):204-225 (1994); Stout et al., “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Chemtracts: Org. Chem., 8(6):359-362 (1995), or TS-962 (Taisho Pharmaceutical Co. Ltd.).

The hypolipidemic agent may be an up-regulator of LD2 receptor activity, such as MD-700 (Taisho Pharmaceutical Co. Ltd) and LY295427 (Eli Lilly).

Examples of suitable cholesterol absorption inhibitor for use in combination with the compound of the invention include SCH48461 (Schering-Plough), as well as those disclosed in Atherosclerosis, 115:45-63 (1995) and J. Med. Chem., 41:973 (1998).

Examples of suitable ileal Na⁺/bile acid co-transporter inhibitors for use in combination with the compound of the invention include compounds as disclosed in Drugs of the Future, 24:425-430 (1999).

The lipoxygenase inhibitors which may be employed in combination the compound of formula I include 15-lipoxygenase (15-LO) inhibitors, such as benzimidazole derivatives, as disclosed in WO 97/12615, 15-LO inhibitors, as disclosed in WO 97/12613, isothiazolones, as disclosed in WO 96/38144, and 15-LO inhibitors, as disclosed by Sendobry et al., “Attenuation of diet-induced atherosclerosis in rabbits with a highly selective 15-lipoxygenase inhibitor lacking significant antioxidant properties”, Brit. J. Pharmacology, 120:1199-1206 (1997), and Cornicelli et al., “15-Lipoxygenase and its Inhibition: A Novel Therapeutic Target for Vascular Disease”, Current Pharmaceutical Design, 5:11-20 (1999).

Examples of suitable anti-hypertensive agents for use in combination with the compound of the present invention include beta adrenergic blockers, calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable anti-obesity agents for use in combination with the compound of the present invention include a beta 3 adrenergic agonist, a lipase inhibitor, a serotonin (and dopamine) reuptake inhibitor, a thyroid receptor beta drug, 5HT2C agonists, (such as Arena APD-356); MCHR1 antagonists such as Synaptic SNAP-7941 and Takeda T-226926, melanocortin receptor (MC4R) agonists, melanin-concentrating hormone receptor (MCHR) antagonists (such as Synaptic SNAP-7941 and Takeda T-226926), galanin receptor modulators, orexin antagonists, CCK agonists, NPY1 or NPY5 antagonist, NPY2 and NPY4 modulators, corticotropin releasing factor agonists, histamine receptor-3 (H3) modulators, 11-beta-HSD-1 inhibitors, adinopectin receptor modulators, monoamine reuptake inhibitors or releasing agents, a ciliary neurotrophic factor (CNTF, such as AXOKINE® by Regeneron), BDNF (brain-derived neurotrophic factor), leptin and leptin receptor modulators, cannabinoid-1 receptor antagonists (such as SR-141716 (Sanofi) or SLV-319 (Solvay)), and/or an anorectic agent.

The beta 3 adrenergic agonists which may be optionally employed in combination with compound of the present invention include AJ9677 (Takeda/Dainippon), L750355 (Merck), or CP331648 (Pfizer,) or other known beta 3 agonists, as disclosed in U.S. Pat. Nos. 5,541,204, 5,770,615, 5,491,134, 5,776,983 and 5,488,064.

Examples of lipase inhibitors which may be optionally employed in combination with compound of the present invention include orlistat or ATL-962 (Alizyme).

The serotonin (and dopamine) reuptake inhibitor (or serotonin receptor agonists) which may be optionally employed in combination with a compound of the present invention may be BVT-933 (Biovitrum), sibutramine, topiramate (Johnson & Johnson) or axokine (Regeneron).

Examples of thyroid receptor beta compounds which may be optionally employed in combination with the compound of the present invention include thyroid receptor ligands, such as those disclosed in WO 97/21993 (U. Cal SF), WO 99/00353 (KaroBio) and WO 00/039077 (KaroBio).

The monoamine reuptake inhibitors which may be optionally employed in combination with compound of the present invention include fenfluramine, dexfenfluramine, fluvoxamine, fluoxetine, paroxetine, sertraline, chlorphentermine, cloforex, clortermine, picilorex, sibutramine, dexamphetamine, phentermine, phenylpropanolamine or mazindol.

The anorectic agent which may be optionally employed in combination with the compound of the present invention include topiramate (Johnson & Johnson), dexamphetamine, phentermine, phenylpropanolamine or mazindol.

The aforementioned patents and patent applications are incorporated herein by reference.

The above other therapeutic agents, when employed in combination with the compound of the present invention may be used, for example, in those amounts indicated in the Physicians' Desk Reference, as in the patents set out above or as otherwise determined by one of ordinary skill in the art.

What is claimed is:

1. A crystal structure of

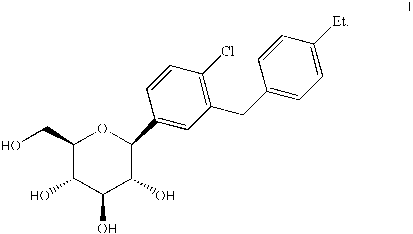

2. The crystal structure according to claim 1 comprising a structure selected from the group consisting of H-1, H-2 and (S)—PG.

3. The crystal structure of claim 1 comprising a structure selected from the group consisting of H-2 and (S)—PG.

4. The crystal structure of claim 3 wherein said structure is in substantially pure form.

5. The crystal structure of the (S)-propylene glycol ((S)—PG) Ia

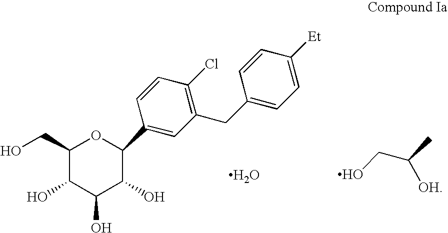

6. The crystal structure of the L-phenylalanine (L-Phe) (form H-1) Ib

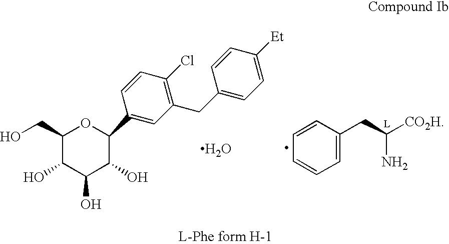

7. The crystal structure of the L-phenylalanine (L-Phe) (form H-2) Ic

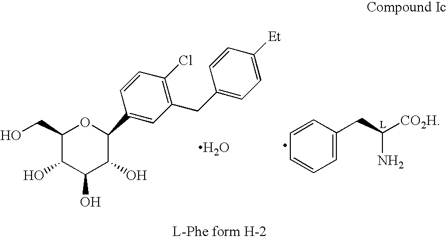

8. The crystal structure H-2 according to claim 1 characterized by one or more of the following:
a) unit cell parameters substantially equal to the following:
Cell dimensions:
a=11.564(4) Å
b=5.954(1) Å
c=21.123(6) Å
α=−degrees
β=96.96(2) degrees
γ=degrees
Space group P2₁
Molecules/asymmetric unit 1
wherein measurement of said crystal structure is at a temperature of about 25° C., and which is characterized by fractional atomic coordinates substantially as listed in Table 5;
b) a powder x-ray diffraction pattern comprising 2Θ values (CuKα λ=1.5418 Å) selected from the group consisting of 4.2±0.1, 8.3±0.1, 9.2±0.1, 10.7±0.1, 15.5±0.1, 18.4±0.1, 19.2±0.1 and 21.6±0.1, at room temperature or as shown in FIG. 1;
c) a differential scanning calorimetry thermogram having an endotherm in the range of about RT to 110° C. or as shown in FIG. 3;
d) thermal gravimetric analysis curve with about 3.1% weight loss up to about 110° C. or as shown in FIG. 5; or
e) a moisture sorption isotherm with <0.3% water uptake in the range 25-75% relative humidity at 25° C. or as shown in FIG. 7.

9. The crystal structure H-1 according to claim 1 characterized by the following:
unit cell parameters substantially equal to the following:
Cell dimensions at 22° C.:
a=11.738(3)Å
b=5.882(1)Å
c=21.001(4)Å
α=−degrees
β=86.66(1) degrees
γ=−degrees
Space group P2₁
Molecules/asymmetric unit 1
wherein measurement of said crystal structure is at a temperature of about 22° C. and which is characterized by, fractional atomic coordinates substantially as tested in Table 6.

10. The crystal structure (S—PG) according to claim 1 characterized by one or more of the following:
a) input unit cell parameters substantially equal to the following:
Cell dimensions:
a=11.269 Å
b=4.809 Å
c=46.723 Å
α=90.0 degrees
β=90.0 degrees
γ=90.0 degrees
Space group P2₁2₁2₁
Molecules/asymmetric unit 1
wherein measurement of said crystal structure is at a temperature of about room temperature;
b) hybrid (refined) unit cell parameters substantially equal to the following:
Cell dimensions:
a=11.219 Å
b=4.782 Å
c=47.124 Å
α=90.0 degrees
β=90.0 degrees
γ=90.0 degrees
Space group P2₁2₁2₁
Molecules/asymmetric unit 1
wherein measurement of said crystal structure is at a temperature of about room temperature;
c) a powder x-ray diffraction pattern comprising 2Θ values (CuKαλ=1.5418 Å) selected from the group consisting of 3.7±0.1, 8.1±0.1, 8.7±0.1, 15.0±0.1, 15.8±0.1, 17.0±0.1, 18.9±0.1, 20.2±0.1 and 21.8±0.1, at room temperature or as shown in FIG. 2;
d) a differential scanning calorimetry thermogram having an endotherm in the range of about RT to 70° C. or as shown in FIG. 4;
e) a thermal gravimetric analysis curve with weight loss of about 3.7% up to about 70° C., and weight loss of about 19.3% at up to about 220° C. or as shown in FIG. 6; or
f) a solid state ¹³C NMR spectrum having substantially similar peak positions at 14.1, 18.1, 27.0, 39.6, 61.1, 69.9, 76.7, 78.5, 78.9, 124.0, 131.5, 136.3 and 141.0, as determined on a 400 MHz spectrometer relative to TMS at zero.

11. A process for preparing the compound of Formula Ia as defined in claim 5 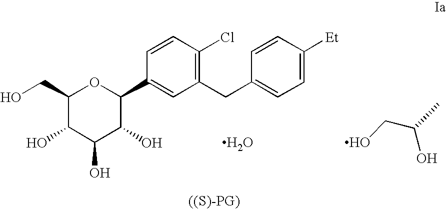
which comprises:
reacting a compound of Formula I 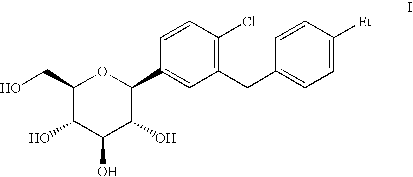
in an organic solvent with (S)-propylene glycol to yield the compound of Formula Ia.

12. The process as defined in claim 11 including the step of adding seeds of compound Ia ((S)—PG) to the reaction mixture containing compound I and (S)-propylene glycol.

13. The process as defined in claim 11 wherein the organic solvent is an alkyl acetate or methyl t-butyl ether.

14. A process for preparing a crystalline compound Ia as defined in claim 5, which comprises reacting a compound of the structure 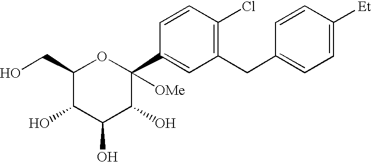
with a reducing agent in the presence of an activating group to form compound I of the structure 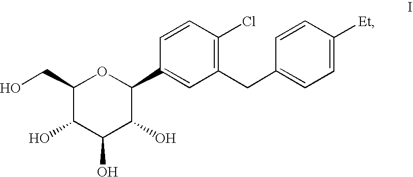
reacting compound I with (S)-propylene glycol in the presence of an organic solvent, optionally adding seeds of compound Ia ((S)—PG) to the reaction mixture, to form crystalline compound Ia

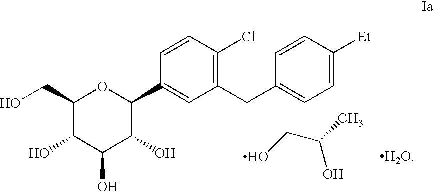

15. The process as defined in claim 14 wherein the reducing agent is an alkylsulfyl halide and the activating group is a Lewis acid.

16. The process as defined in claim 15 wherein the reducing agent is triethylsilane, the activating group is BF₃OEt₂ or BF₃.2CH₃COOH, and in the organic solvent is methyl t-butyl ether.

17. A process for preparing the L-phenylalanine of the structure of formula Ic form H-2 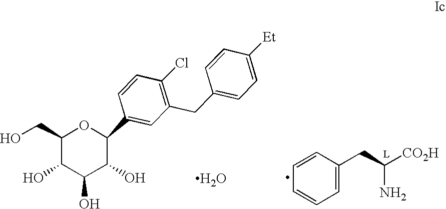
which comprises
a) providing compound A of the structure 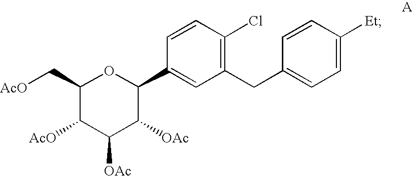
b) dissolving compound A in an alcohol solvent;
c) treating the resulting solution with strong base;
d) heating the resulting mixture at a temperature within the range from about 45 to about 55° C.;
e) cooling the resulting mixture to a temperature within the range from about 15 to about 30° C.;
f) adding strong mineral acid to the cooled mixture to neutralize the mixture;
g) adding L-phenylalanine in an organic solvent and water to the mixture;
h) heating the mixture to obtain a solution;
i) optionally adding seeds of compound Ic form H-2, in a slurry with water and solvent, to the solution; and
j) cooling the resulting solution to form crystals of compound Ic form H-2.

18. The process as defined in claim 17 wherein the organic solvent employed is isopropyl alcohol.

19. The process as defined in claim 17 including the step of recovering crystals of compound Ib form H-1 of the structure

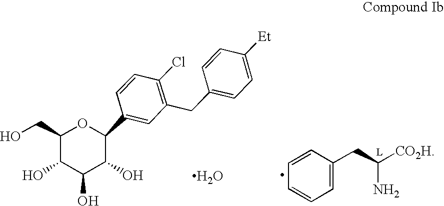

20. A pharmaceutical composition comprising an effective amount of the crystal structure according to claim 1 and a pharmaceutically acceptable carrier or diluent.

21. The pharmaceutical composition according to claim 20 wherein said crystal structure is selected from the group consisting of H-2 and S—PG.

22. The pharmaceutical composition according to claim 20 wherein said crystal structure is in substantially pure form.

23. A pharmaceutical composition comprising an effective amount of the crystal structure according to claim 1 in combination with one or more therapeutic agents selected from the group consisting of an antidiabetic agent, an anti-obesity agent, a anti-hypertensive agent, an anti-atherosclerotic agent and a lipid-lowering agent.

24. The pharmaceutical composition according to claim 23 wherein said crystal structure is selected from H-2 and S—PG.

25. The pharmaceutical composition according to claim 24 wherein said crystal structure is in substantially pure form.

26. A method of treating diabetes, diabetic retinopathy, diabetic neuropathy, diabetic nephropathy, delayed wound healing, insulin resistance, hyperglycemia, hyperinsulinemia, elevated blood levels of fatty acids or glycerol, dyslipidemia, hyperlipidemia, obesity, hypertriglyceridemia, Syndrome X, diabetic complications, atherosclerosis or hypertension, or for increasing high density lipoprotein levels in a mammal comprising administering to the mammal a therapeutically-effective amount of the crystal structure according to claim 5.

27. A method of treating diabetes, diabetic retinopathy, diabetic neuropathy, diabetic nephropathy, delayed wound healing, insulin resistance, hyperglycemia, dyslipidemia, hyperinsulinemia, elevated blood levels of fatty acids or glycerol, hyperlipidemia, obesity, hypertriglyceridemia, Syndrome X, diabetic complications, atherosclerosis or hypertension, or for increasing high density lipoprotein levels in a mammal comprising administering to the mammal a therapeutically-effective amount of the crystal structure according to claim 7.

28. The method according to claim 27 wherein the mammal is a human.
